(19) United States
(12) Patent Application Publication
(10) Pub. No.: US 2022/0282320 A1
(43) Pub. Date: Sep. 8, 2022

(54) PHOTO-TRIGGERED NUCLEIC ACID CONSTRUCTS AND METHODS FOR MOLECULAR DETECTION

(71) Applicant: InSilixa, Inc., Sunnyvale, CA (US)

(72) Inventors: Arjang Hassibi, Santa Clara, CA (US); Robert G. Kuimelis, Palo Alto, CA (US); Lei Pei, San Jose, CA (US); Kirsten A. Johnson, Redwood City, CA (US); Jessica C. Ebert, Mountain View, CA (US); Arun Manickam, San Jose, CA (US); Tran T. Van, San Jose, CA (US)

(21) Appl. No.: 17/530,932

(22) Filed: Nov. 19, 2021

Related U.S. Application Data
(63) Continuation of application No. PCT/US2020/033884, filed on May 20, 2020.
(60) Provisional application No. 62/850,239, filed on May 20, 2019.

(51) Int. Cl. C12Q 1/6853

(52) U.S. Cl. CPC C12Q 1/6853; C12Q 1/6851

(57) ABSTRACT

The present disclosure provides methods, devices and systems that enable simultaneous multiplexing amplification reaction and real-time detection in a single reaction chamber.

### CROSS REFFERENCE

This application is a continuation of International Application No. PCT/US2020/033884, filed May 20, 2020, which claims priority to U.S. Provisional Patent Application No. 62/850,239, filed May 20, 2019, which are herein incorporated by reference in their entirety for all purposes.

### SEQUENCE LISTING

The instant application contains a Sequence Listing which has been submitted in ASCII format via EFS-Web and is hereby incorporated by reference in its entirety. Said ASCII copy, created Nov. 15, 2021, is named 42500_725_301 SL.txt and is 3,386 bytes in size.

### BACKGROUND

Nucleic acid (NA) tests are unique analytical techniques used to detect, quantify, and identify the genetic structure of specific sequences of deoxyribonucleic acid (DNA) or ribonucleic acid (RNA) molecules. NA tests have many applications and are widely used in both life-science research and molecular diagnostics. Independent of the application and the testing venue, the amount of genetic material (RNA or DNA copies) in the testing sample is typically very small and not directly detectable; therefore, it is very common to use physiochemical, biochemical, or enzymatic methods to enhance the generated target-specific signals to ensure more sensitive tests. Some of these methods utilize molecular amplification processes such as polymerase chain reaction (PCR) to increase the copy number of the target NA. Such tests are categorized and are widely known and are conventionally categorized as nucleic acid amplification tests (NAATs). In addition, methods of amplification include, for example: strand displacement amplification (SDA), and nucleic acid sequence based amplification (NASBA), and Rolling Circle Amplification (RCA).

NAATs methods have a variety of different performance criteria which include analytical sensitivity, specificity, limit of detection (LoD), quantification range, detection dynamic range (DDR), and turnaround time (TAT). Different applications call for different criteria and there are always tradeoffs, depending of the method used. For example, in infectious disease application, it is critical to accurately identify the presence or absence of the infecting pathogen in the clinical specimen. Therefore, one requires NAAT methods that offer LOD of a few organisms per test, while the quantification range is less critical as the patient treatment is less reliant on that information. On the other hand, in gene expression applications, the concentration of messenger RNA (mRNA) in the clinical sample is relatively large and DDR is much more important than LOD.

Today, there are variety of NAAT methods for NA detection which use specific enzymes, reagents, and temperature profiles to amplify and detect specific sequences. In this invention, we describe methods and molecular structures, that once included in specific NAAT methods, can improve their performance criteria.

### SUMMARY

In this invention, unique nucleic acid (NA) construct and methods are described that by their incorporation into molecular detection assays, one can improve the assay detection performance, broadly defined, and reduce the workflow complexity and its turnaround time.

Aspects of the present disclosure provide a reaction chamber, comprising: a NA construct comprising a photosensitive chemical moiety, wherein the NA construct is in a first molecular state, wherein the NA construct is configured to change to a second molecular state after exposure to a light; at least one reagent; and at least one enzyme; wherein the reaction chamber is configured to allow the light to reach the nucleic acid construct.

In some embodiments of aspects provided herein, the NA construct is an oligonucleotide primer or a probe. In some embodiments of aspects provided herein, the at least one enzyme is a polymerase, reverse transcriptase, a terminal transferase, an exonuclease, an endonuclease, a restriction enzyme, or a ligase. In some embodiments of aspects provided herein, the wherein the at least one reagent comprises one or more amplification reagents. In some embodiments of aspects provided herein, the method further comprises a target NA. In some embodiments of aspects provided herein, the enzyme is configured to catalyze a reaction associated with the target NA, the at least one reagent and the NA construct. In some embodiments of aspects provided herein, the NA construct in the first molecular state is configured to be active in the reaction. In some embodiments of aspects provided herein, the NA construct in the second molecular state is configured to be inactive in the reaction. In some embodiments of aspects provided herein, the NA construct in the first molecular state is configured to be inactive in the reaction. In some embodiments of aspects provided herein, the NA construct in the second molecular state is configured to be active in the reaction. In some embodiments of aspects provided herein, the method further comprising another NA construct comprising another photosensitive chemical moiety, wherein the another NA construct is in a third molecular state, wherein the another NA construct is configured to change to a fourth molecular state after exposure to another light. In some embodiments of aspects provided herein, the another light is the light. In some embodiments of aspects provided herein, the NA construct in the first molecular state is configured to be active in the reaction and another NA construct in the third molecular state is configured to be inactive in the reaction. In some embodiments of aspects provided herein, the NA construct in the second molecular state is configured to be inactive in the reaction and the another NA construct in the fourth molecular state is configured to be active in the reaction. In some embodiments of aspects provided herein, the NA construct in the first molecular state and the another NA construct in the third molecular state are configured to be active in the reaction. In some embodiments of aspects provided herein, the NA construct in the second molecular state and the other NA construct in the fourth molecular state are configured to be inactive in the reaction. In some embodiments of aspects provided herein, the NA construct in the first molecular state and the another NA construct in the third molecular state are configured to be inactive in the reaction.

In some embodiments of aspects provided herein, the NA construct in the second molecular state and the another NA construct in the fourth molecular state are configured to be active in the reaction. In some embodiments of aspects provided herein, the enzyme is the polymerase, the reaction is polymerase chain reaction, and the NA construct is the oligonucleotide primer. In some embodiments of aspects provided herein, the photosensitive chemical moiety locates at 3′-terminus, at 5′-terminus, or in the middle of the NA construct. In some embodiments of aspects provided herein, the NA construct further comprises an additional photosensitive chemical moiety. In some embodiments of aspects provided herein, the fifth molecular state is the first molecular state, and the sixth molecular state is the second molecular state. In some embodiments of aspects provided herein, the reaction chamber is a closed-tube reaction chamber.

Another aspect of the present disclosure provides a of conducting a reaction, comprising: activating a reaction chamber to conduct a reaction, the reaction chamber comprising: a nucleic acid construct comprising a photosensitive chemical moiety in a first molecular state; at least one reagent; and at least one enzyme; and activating a light to reach the nucleic acid construct in the reaction chamber, thereby changing the nucleic acid construct to a second molecular state.

In some embodiments of aspects provided herein, the NA construct is an oligonucleotide primer or a probe. In some embodiments of aspects provided herein, the at least one enzyme is a polymerase, reverse transcriptase, a terminal transferase, an exonuclease, an endonuclease, a restriction enzyme, or a ligase. In some embodiments of aspects provided herein, the at least one reagent comprises one or more amplification reagents. In some embodiments of aspects provided herein, the reaction chamber further comprises a target nucleic acid. In some embodiments of aspects provided herein, the enzyme catalyzes the reaction of the target nucleic acid with the at least one reagent and the nucleic acid construct. In some embodiments of aspects provided herein, the nucleic acid construct in the first molecular state is active in the reaction. In some embodiments of aspects provided herein, the nucleic acid construct in the first molecular state is inactive in the reaction. In some embodiments of aspects provided herein, the nucleic acid construct in the second molecular state is active in the reaction. In some embodiments of aspects provided herein, the reaction chamber further comprises another nucleic acid construct comprising another photosensitive chemical moiety in a third molecular state, wherein the another nucleic acid construct is configured to change to a fourth molecular state after exposure to another light. In some embodiments of aspects provided herein, the another light is the light, and wherein the activating the light activates the nucleic acid construct. In some embodiments of aspects provided herein, the method further comprises: activating the another light to reach the another nucleic acid construct. In some embodiments of aspects provided herein, the method further comprises: deactivating the nucleic acid construct in the reaction after the activating the light. In some embodiments of aspects provided herein, the method further comprises: activating the another nucleic acid construct after the activating the light or after the activating the another light. In some embodiments of aspects provided herein, the method further comprises: deactivating the another nucleic acid construct after the activating the light or the activating the another light. In some embodiments of aspects provided herein, the method further comprises: activating the nucleic acid construct in the reaction after the activating the light. In some embodiments of aspects provided herein, the method further comprises: deactivating the another nucleic acid construct after the activating the light or the activating the another light. In some embodiments of aspects provided herein, the method further comprises: activating the another nucleic acid construct after the activating the light or the activating the another light. In some embodiments of aspects provided herein, the reaction is extension, digest, transcription, terminal transfer, or ligation. In some embodiments of aspects provided herein, when conducting the reaction in the reaction chamber, no external reagents are added into the reaction chamber In some embodiments of aspects provided herein, when conducting the reaction in the reaction chamber, none of the nucleic acid construct, the enzyme is the polymerase, the reaction is a polymerase chain reaction, and the nucleic acid construct is the oligonucleotide primer. at least one reagent, or the at least one enzyme are removed from the reaction chamber. In some embodiments of aspects provided herein, the photosensitive chemical moiety locates at 3-terminus, at 5-terminus, or in the middle of the nucleic acid construct. In some embodiments of aspects provided herein, the nucleic acid construct further comprises an additional photosensitive chemical moiety. In some embodiments of aspects provided herein, the target nucleic acid comprises a major allele and a minor allele, and wherein the reaction is polymerase chain reaction. In some embodiments of aspects provided herein, the nucleic acid construct comprises a sequence complementary to the major allele. In some embodiments of aspects provided herein, the nucleic acid construct in the first molecular state is inactive in the polymerase chain reaction with regard to making an amplicon of the major allele. In some embodiments of aspects provided herein, the nucleic acid construct in the second molecular state is active in the polymerase chain reaction with regard to making the amplicon of the major allele. In some embodiments of aspects provided herein, the nucleic acid construct in the second molecular state is inactive in the polymerase chain reaction with regard to making the amplicon of the major allele. In some embodiments of aspects provided herein, the another nucleic acid construct is a primer for the minor allele, and the method further comprises producing amplicons of the minor allele before the activating the light.

Aspects of the present disclosure provide a nucleic acid construct, comprising: a) a plurality of nucleotides; and b) one or more photocleavable moieties; wherein each of the one or more photocleavable moieties is independently located: a) at 3′-terminus of the nucleic acid construct; b) at 5′-terminus of the nucleic acid construct; c) between the 3′-terminus and the 5′-terminaus; d) on or connected to a nucleobase; e) on or connected to a ribose; f) between and connected to two consecutive members of the plurality of nucleotides; or g) a combination thereof

In some embodiments of aspects provided herein, the nucleic acid construct is configured to be inactive in a biochemical reaction, wherein the biochemical reaction is polymerase-catalyzed chain elongation, polymerase chain reaction (PCR), reverse transcription polymerase chain reaction (RT-PCR), ligation, terminal transferases extension, hybridizations, exonuclease digest, endonuclease digest, or restriction digest. In some embodiments of aspects provided herein, the nucleic acid construct is configured to form a nucleic acid molecule after photocleavage of the one or more photocleavable moieties, and wherein the nucleic acid molecule is configured to be active in the biochemical reaction. In some embodiments of aspects provided herein, the nucleic acid construct is a primer, and wherein the biochemical reaction is polymerase-catalyzed chain elongation. In some embodiments of aspects provided herein, the one or more photocleavable moieties are located at the 3′-terminus. In some embodiments of aspects provided herein, each of the one or more photocleavable moieties is independently located between the 3′-terminus and the 5′-terminaus and on a selected nucleobase. In some embodiments of aspects provided herein, each of the one or more photocleavable moieties is independently located between the 3′-terminus and the 5′-terminaus and between the two consecutive members of the plurality of nucleotides. In some embodiments of aspects provided herein, the 3′-terminus is configured to be inactive in the biochemical reaction. In some embodiments of aspects provided herein, the nucleic acid construct comprises a first nucleic acid section and a second nucleic acid section complementary to the first nucleic acid section, wherein the nucleic acid construct is configured to form a hairpin structure. In some embodiments of aspects provided herein, the first nucleic acid section and the second nucleic acid section do not comprise the one or more photocleavable moieties.

Aspects of the present disclosure provide a method of conducting the polymerase-catalyzed chain elongation using the nucleic acid construct of the present disclosure, comprising: a) providing a reaction mixture comprising the nucleic acid construct, at least one template nucleic acid molecule, a polymerase, wherein the nucleic acid construct has sequence complementary with the template nucleic acid molecule; b) subjecting the reaction mixture to conditions for the polymerase-catalyzed chain elongation; and c) radiating the reaction mixture or the nucleic acid construct with photons of light, thereby performing the polymerase-catalyzed chain elongation.

In some embodiments of aspects provided herein, the subjecting in b) does not enable the performing in c). In some embodiments of aspects provided herein, the nucleic acid construct remains intact in the reaction mixture before the radiating in c). In some embodiments of aspects provided herein, the method further comprises: in c), cleaving the one or more photocleavable moieties. In some embodiments of aspects provided herein, the method further comprises: in c), forming the nucleic acid molecule. In some embodiments of aspects provided herein, the performing in c) comprises using the nucleic acid molecule formed in c) after the radiating as a primer for the polymerase-catalyzed chain elongation. In some embodiments of aspects provided herein, the reaction mixture further comprises another primer, wherein the another primer is active in the polymerase-catalyzed chain elongation. In some embodiments of aspects provided herein, the another primer is active in the polymerase-catalyzed chain elongation before the radiating in c). In some embodiments of aspects provided herein, the polymerase-catalyzed chain elongation in b) produces an amplicon comprising the another primer. In some embodiments of aspects provided herein, the polymerase-catalyzed chain elongation is a quantitative polymerase chain reaction (Q-PCR), the method further comprises: in c), 1) performing the polymerase-catalyzed chain elongation on two or more nucleotide sequences in the presence of the nucleic acid construct of the present disclosure to produce two or more amplicons in a fluid; 2) providing an array comprising a solid surface with a plurality of nucleic acid probes at independently addressable locations, the array configured to contact the fluid; and 3) measuring hybridization of the two or more amplicons to two or more nucleic acid probes of the plurality of nucleic acid probes while the fluid is in contact with the array to obtain an amplicon hybridization measurement, wherein the amplicons comprise a quencher. In some embodiments of aspects provided herein, the polymerase-catalyzed chain elongation is a quantitative polymerase chain reaction (Q-PCR), the method further comprises: in c), 1) providing an array comprising a solid support having a surface and a plurality of different probes, the plurality of different probes immobilized to the surface at different addressable locations, each addressable location comprising a fluorescent moiety; 2) performing PCR amplification on a sample comprising a plurality of nucleotide sequences; the PCR amplification carried out in a fluid, wherein:(i) the nucleic acid construct of the present disclosure is a PCR primer for each nucleic acid sequence and comprises a quencher; and (ii) the fluid is in contact with the plurality of different probes, wherein amplicons produced in the PCR amplification hybridize with the plurality of probes, thereby quenching signal from the fluorescent moiety; 3) detecting the signal from the fluorescent moiety at each of the addressable locations over time; 4) using the signal detected over time and determining an amount of the amplicons in the fluid; and 5) using the amount of the amplicons in the fluid to determine an amount of the nucleotide sequences in the sample. In some embodiments of aspects provided herein, the polymerase-catalyzed chain elongation is a quantitative polymerase chain reaction (Q-PCR), the method further comprises: in c): 1) providing the reaction mixture comprising a nucleic acid sample containing at least one template nucleic acid molecule, a primer pair and a polymerase, wherein the primer pair has sequence complementarity with the template nucleic acid molecule, and wherein the primer pair comprises a limiting primer and an excess primer, wherein at least one of the limiting primer and the excess primer is the nucleic acid construct of the present disclosure; 2) subjecting the reaction mixture to the Q-PCR under conditions that are sufficient to yield at least one target nucleic acid molecule as an amplification product of the template nucleic acid molecule and the limiting primer, which at least one target nucleic acid molecule comprises the limiting primer; 3) bringing the reaction mixture in contact with a sensor array having (i) a substrate comprising a plurality of probes immobilized to a surface of the substrate at different individually addressable locations, wherein the probes have sequence complementarity with the limiting primer and are capable of capturing the limiting primer, and (ii) an array of detectors configured to detect at least one signal from the addressable locations, wherein the at least one signal is indicative of the limiting primer binding with an individual probe of the plurality of probes; 4) using the array of detectors to detect the at least one signal from one or more the addressable locations at multiple time points during the nucleic acid amplification reaction; and 5) detecting the target nucleic acid molecule based on the at least one signal indicative of the limiting primer binding with the individual probe of the plurality of probes.

Aspects of the present disclosure provides a system for assaying at least one target nucleic acid molecule using the nucleic acid construct of the present disclosure, comprising: 1) a reaction chamber comprising a reaction mixture comprising a nucleic acid sample containing at least one template nucleic acid molecule, a primer pair that has sequence complementary to the template nucleic acid molecule, and a polymerase, wherein the primer pair comprises a limiting primer and an excess primer, wherein at least one of the limiting primer and the excess primer is the nucleic acid construct of the present disclosure, wherein the reaction chamber comprising the reaction mixture is configured to facilitate a nucleic acid amplification reaction on the reaction mixture to yield at least one target nucleic acid molecule as an amplification product of the template nucleic acid; 2) a sensor array comprising (i) a substrate comprising a plurality of probes immobilized to a surface of the substrate at different individually addressable locations, wherein the probes have sequence complementarity with the limiting primer and are capable of capturing the limiting primer; and (ii) an array of detectors configured to detect at least one signal from the addressable locations, wherein the at least one signal is indicative of the limiting primer binding with an individual probe of the plurality of probes; and 3) a computer processor coupled to the sensor array and programmed to (i) subject the reaction mixture to the nucleic acid amplification reaction, and (ii) detect the at least one signal from one or more of the addressable locations at multiple time points during the nucleic acid amplification reaction.

Aspects of the present disclosure provides nucleic acid construct, comprising: a) a plurality of nucleotides; and b) one or more photocleavable moieties; wherein each of the one or more photocleavable moieties is independently located: a) between 3′-terminus of the nucleic acid construct and 5′-terminaus of the nucleic acid construct; b) on or connected to a nucleobase; c) on or connected to a ribose; d) between and connected to two consecutive members of the plurality of nucleotides; or e) a combination thereof.

In some embodiments of aspects provided herein, the nucleic acid construct is configured to be active in a biochemical reaction, wherein the biochemical reaction is polymerase-catalyzed chain elongation, polymerase chain reaction (PCR), reverse transcription polymerase chain reaction (RT-PCR), ligation, terminal transferases extension, hybridizations, exonuclease digest, endonuclease digest, or restriction digest. In some embodiments of aspects provided herein, the nucleic acid construct is configured to form a nucleic acid molecule after photocleavage of the one or more photocleavable moieties, and wherein the nucleic acid molecule is inactive in the biochemical reaction. In some embodiments of aspects provided herein, the nucleic acid construct is configured to form a nucleic acid molecule near after photocleavage of the one or more photocleavable moieties, and wherein the nucleic acid molecule is active in the biochemical reaction, wherein the nucleic acid molecule locates near the 3′-terminus. In some embodiments of aspects provided herein, the nucleic acid construct is a primer, and wherein the biochemical reaction is polymerase-catalyzed chain elongation. In some embodiments of aspects provided herein, each of the one or more photocleavable moieties is independently located between the 3′-terminus and the 5′-terminaus and on a selected nucleobase. In some embodiments of aspects provided herein, the nucleic acid construct is configured to form a hairpin structure in the absence of the one or more photocleavable moieties, thereby rendered inactive as the primer in the absence of the one or more photocleavable moieties. In some embodiments of aspects provided herein, each of the one or more photocleavable moieties is independently located between the 3′-terminus and the 5′-terminaus and between the two consecutive members of the plurality of nucleotides. In some embodiments of aspects provided herein, the nucleic acid construct comprise a first sequence complimentary to a template nucleic acid molecule, and wherein the first sequence locates at or near the 3′-terminus. In some embodiments of aspects provided herein, the nucleic acid construct further comprises a second sequence complimentary to the template nucleic acid molecule, wherein the second sequence locates at or near the 5′-terminus, and wherein at least one of the one or more photocleavable moieties locates between the first sequence and the second sequence. In some embodiments of aspects provided herein, the one or more photocleavable moieties is separated from the first sequence and/or the second sequence by at least one nucleotide. In some embodiments of aspects provided herein, the nucleic acid construct is configured to form a hairpin loop between the first sequence and the second sequence when both the first sequence and the second sequence hybridize with the template nucleic acid molecule. In some embodiments of aspects provided herein, the second sequence comprises connects with a 5′ to 5′ linkage to rest of the nucleic acid construct, and wherein the second sequence is configured to be non-extensible in the polymerase-catalyzed chain elongation.

Aspects of the present disclosure provide a method of conducting the polymerase-catalyzed chain elongation using the nucleic acid construct of the present disclosure, comprising: a) providing a reaction mixture comprising the nucleic acid construct, the template nucleic acid molecule, a polymerase, wherein the nucleic acid construct comprises at least the first sequence; b) subjecting the reaction mixture to conditions for the polymerase-catalyzed chain elongation, thereby performing the polymerase-catalyzed chain elongation; and c) radiating the reaction mixture or the nucleic acid construct with photons of light, thereby stopping the polymerase-catalyzed chain elongation.

In some embodiments of aspects provided herein, the method further comprises: in c), cleaving the one or more photocleavable moieties. In some embodiments of aspects provided herein, the method further comprises: in c), forming the nucleic acid molecule after the radiating, wherein the nucleic acid molecule dissociate from the template nucleic acid molecule. In some embodiments of aspects provided herein, the nucleic acid molecule forms a hairpin structure, and wherein the hairpin structure comprises at least part of the first sequence. In some embodiments of aspects provided herein, the nucleic acid molecule comprises the first sequence.

Aspects of the present disclosure provide a method of conducting a light-enabled nested polymerase chain reaction (PCR), comprising: a) providing a reaction mixture comprising a first primer pair, a second primer pair, a template nucleic acid molecule comprising an inner nucleic acid sequence, and a polymerase, wherein each member of the first primer pair is independently the nucleic acid construct of the present disclosure, wherein each member of the second primer pair is independently the nucleic acid construct of the present disclosure, wherein the inner nucleic acid sequence is nested within the template nucleic acid molecule; b) subjecting the reaction mixture to conditions for a first chain elongation using the first primer pair to amplify the template nucleic acid molecule, thereby forming amplicons of the template nucleic acid or a complementary sequence of the template nucleic acid molecule; and c) radiating the reaction mixture with photons of light, thereby deactivating the first primer pair and stopping the first elongation, activating the second primer pair and starting a second chain elongation using the activated second primer pair, and forming amplicons of the inner nucleic acid sequence or complementary sequence of the inner nucleic acid sequence, wherein a)-c) are conducted in a closed tube fashion.

In some embodiments of aspects provided herein, the light enabled PCR is a quantitative polymerase chain reaction (Q-PCR), the method further comprises: 1) performing the light enabled PCR on two or more nucleotide sequences in the presence of the first primer pair and second primer pair to produce two or more amplicons in a fluid; 2) providing an array comprising a solid surface with a plurality of nucleic acid probes at independently addressable locations, the array configured to contact the fluid; and 3) measuring hybridization of the two or more amplicons to two or more nucleic acid probes of the plurality of nucleic acid probes while the fluid is in contact with the array to obtain an amplicon hybridization measurement, wherein the amplicons comprise a quencher.

In some embodiments of aspects provided herein, the light enabled PCR is a quantitative polymerase chain reaction (Q-PCR), the method further comprises: 1) providing an array comprising a solid support having a surface and a plurality of different probes, the plurality of different probes immobilized to the surface at different addressable locations, each addressable location comprising a fluorescent moiety; 2) performing PCR amplification on a sample comprising a plurality of nucleotide sequences; the PCR amplification carried out in a fluid, wherein:(i) each of the first pair of primers and the second pair of primer for each nucleic acid sequence comprises a quencher; and (ii) the fluid is in contact with the plurality of different probes, wherein amplicons produced in the PCR amplification hybridize with the plurality of probes, thereby quenching signal from the fluorescent moiety; 3) detecting the signal from the fluorescent moiety at each of the addressable locations over time; 4) using the signal detected over time and determining an amount of the amplicons in the fluid; and 5) using the amount of the amplicons in the fluid to determine an amount of the nucleotide sequences in the sample. In some embodiments of aspects provided herein, the light enabled PCR is a quantitative polymerase chain reaction (Q-PCR), the method further comprises: 1) providing the reaction mixture comprising a nucleic acid sample containing at least one template nucleic acid molecule, a primer pair and a polymerase, wherein the primer pair has sequence complementarity with the template nucleic acid molecule, and wherein the primer pair comprises a limiting primer and an excess primer, wherein at least one of the limiting primer and the excess primer is the nucleic acid construct of the present disclosure; 2) subjecting the reaction mixture to the Q-PCR under conditions that are sufficient to yield at least one target nucleic acid molecule as an amplification product of the template nucleic acid molecule and the limiting primer, which at least one target nucleic acid molecule comprises the limiting primer; 3) bringing the reaction mixture in contact with a sensor array having (i) a substrate comprising a plurality of probes immobilized to a surface of the substrate at different individually addressable locations, wherein the probes have sequence complementarity with the limiting primer and are capable of capturing the limiting primer, and (ii) an array of detectors configured to detect at least one signal from the addressable locations, wherein the at least one signal is indicative of the limiting primer binding with an individual probe of the plurality of probes; 4) using the array of detectors to detect the at least one signal from one or more the addressable locations at multiple time points during the nucleic acid amplification reaction; and 5) detecting the target nucleic acid molecule based on the at least one signal indicative of the limiting primer binding with the individual probe of the plurality of probes.

Aspects of the present disclosure provides a nucleic acid construct, comprising: a) a plurality of nucleotides; and b) one or more photocleavable moieties; wherein each of the one or more photocleavable moieties is independently located: a) between 3′-terminus of the nucleic acid construct and 5′-terminaus of the nucleic acid construct; b) on or connected to a nucleobase; c) on or connected to a ribose; d) between and connected to two consecutive members of the plurality of nucleotides; or e) a combination thereof

In some embodiments of aspects provided herein, the nucleic acid construct is a probe, and wherein the nucleic acid construct is configured to be inactive in hybridization with a target nucleic acid molecule. In some embodiments of aspects provided herein, the nucleic acid construct is configured to form a nucleic acid molecule after photocleavage of the one or more photocleavable moieties, and wherein the nucleic acid molecule is configured to be active in the hybridization with the target nucleic acid molecule. In some embodiments of aspects provided herein, the nucleic acid construct comprises one free end. In some embodiments of aspects provided herein, the nucleic acid construct comprises an immobilized end or an end that is non-extensible in a polymerase-catalyzed chain elongation. In some embodiments of aspects provided herein, each of the one or more photocleavable moieties is independently located between the 3′-terminus and the 5′-terminaus and on a selected nucleobase, wherein the selected nucleobase is configured to hybridize with the target nucleic acid molecule in absence of the one or more photocleavable moieties. In some embodiments of aspects provided herein, each of the one or more photocleavable moieties is independently located between the 3′-terminus and the 5′-terminaus and between the two consecutive members of the plurality of nucleotides. In some embodiments of aspects provided herein, the nucleic acid construct comprises a first nucleic acid section and a second nucleic acid section complementary to the first nucleic acid section, wherein the nucleic acid construct is configured to form a hairpin structure. In some embodiments of aspects provided herein, the first nucleic acid section and the second nucleic acid section do not comprise the one or more photocleavable moieties.

Aspects of the present disclosure provide a method of conducting the hybridization using the nucleic acid construct of the present disclosure, comprising: a) providing a reaction mixture comprising the nucleic acid construct, and the target nucleic acid molecule; b) subjecting the reaction mixture to conditions for the hybridization; and c) radiating the reaction mixture or the nucleic acid construct with photons of light, thereby performing the hybridization.

In some embodiments of aspects provided herein, the subjecting in b) does not enable the performing in c). In some embodiments of aspects provided herein, the nucleic acid construct remains intact in the reaction mixture before the radiating in c). In some embodiments of aspects provided herein, the method further comprises: in c), cleaving the one or more photocleavable moieties. In some embodiments of aspects provided herein, the method further comprises: in c), forming the nucleic acid molecule. In some embodiments of aspects provided herein, the radiating breaks the hairpin structure of the nucleic acid construct and forms the nucleic acid molecule.

Aspects of the present disclosure provide nucleic acid construct, comprising: a) a plurality of nucleotides; and b) one or more photocleavable moieties; wherein each of the one or more photocleavable moieties is independently located: a) between 3′-terminus of the nucleic acid construct and 5′-terminaus of the nucleic acid construct; b) on or connected to a nucleobase; c) on or connected to a ribose; d) between and connected to two consecutive members of the plurality of nucleotides; or e) a combination thereof.

In some embodiments of aspects provided herein, the nucleic acid construct is a probe, and wherein the nucleic acid construct is configured to be active in hybridization with a target nucleic acid molecule. In some embodiments of aspects provided herein, the nucleic acid construct is configured to form a nucleic acid molecule after photocleavage of the one or more photocleavable moieties, and wherein the nucleic acid molecule is configured to be inactive in the hybridization with the target nucleic acid molecule. In some embodiments of aspects provided herein, the nucleic acid construct comprises one free end. In some embodiments of aspects provided herein, the nucleic acid construct comprises an immobilized end or an end that is non-extensible in a polymerase-catalyzed chain elongation. In some embodiments of aspects provided herein, each of the one or more photocleavable moieties is independently located between the 3′-terminus and the 5′-terminaus and between the two consecutive members of the plurality of nucleotides. In some embodiments of aspects provided herein, each of the one or more photocleavable moieties is independently located between the 3′-terminus and the 5′-terminaus and on a selected nucleobase, wherein the selected nucleobase is configured to hybridize with another nucleobase of the nucleic acid construct in absence of the one or more photocleavable moieties. In some embodiments of aspects provided herein, the nucleic acid construct comprises a first nucleic acid section and a second nucleic acid section complementary to the first nucleic acid section, wherein the nucleic acid construct is configured to form a hairpin structure in absence of the one or more photocleavable moieties. In some embodiments of aspects provided herein, the first nucleic acid section or the second nucleic acid section do comprise the one or more photocleavable moieties.

Aspects of the present disclosure provide a method of conducting the hybridization using the nucleic acid construct of the present disclosure, comprising: a) providing a reaction mixture comprising the nucleic acid construct, and the target nucleic acid molecule; b) subjecting the reaction mixture to conditions for the hybridization; and c) radiating the reaction mixture or the nucleic acid construct with photons of light, thereby stopping the hybridization.

In some embodiments of aspects provided herein, the method further comprises: in c), cleaving the one or more photocleavable moieties. In some embodiments of aspects provided herein, the method further comprises: in c), forming the nucleic acid molecule. In some embodiments of aspects provided herein, the method further comprises: in c), forming the hairpin structure in the nucleic acid molecule. In some embodiments of aspects provided herein, the method further comprises conducting a polymerase-catalyzed chain elongation, wherein: 1) the reaction mixture further comprises a polymerase and a primer, wherein in b) the nucleic acid construct hybridize with the target nucleic acid molecule in b); 2) subjecting the reaction mixture in b) to conditions for the polymerase-catalyzed chain elongation using the primer, wherein the polymerase-catalyzed chain elongation stalls at or near a position from which the nucleic acid construct forms a duplex with the target nucleic acid molecule; and 3) after the radiating in c), removing the duplex and exposing a single-stranded sequence previously hybridized with the nucleic acid construct, thereby allowing polymerase-catalyzed chain elongation to continue and elongate through the single-stranded sequence. In some embodiments of aspects provided herein, the polymerase-catalyzed chain elongation is a quantitative polymerase chain reaction (Q-PCR), the method further comprises: 1) performing the polymerase-catalyzed chain elongation on two or more nucleotide sequences comprising the target nucleic acid molecule in the presence of the nucleic acid construct of the present disclosure, thereby producing two or more amplicons in a fluid; 2) providing an array comprising a solid surface with a plurality of nucleic acid probes at independently addressable locations, the array configured to contact the fluid; and 3) measuring hybridization of the two or more amplicons to two or more nucleic acid probes of the plurality of nucleic acid probes while the fluid is in contact with the array to obtain an amplicon hybridization measurement, wherein the amplicons comprise a quencher. In some embodiments of aspects provided herein, the polymerase-catalyzed chain elongation is a quantitative polymerase chain reaction (Q-PCR), the method further comprises: 1) providing an array comprising a solid support having a surface and a plurality of different probes, the plurality of different probes immobilized to the surface at different addressable locations, each addressable location comprising a fluorescent moiety; 2) performing PCR amplification on a sample comprising a plurality of nucleotide sequences comprising the target nucleic acid molecule; the PCR amplification carried out in a fluid comprising the nucleic acid construct of the present disclosure, wherein:(i) a PCR primer for each nucleic acid sequence comprises a quencher; and (ii) the fluid is in contact with the plurality of different probes, wherein amplicons produced in the PCR amplification hybridize with the plurality of probes, thereby quenching signal from the fluorescent moiety; wherein the radiating occurs during the PCR; 3) detecting the signal from the fluorescent moiety at each of the addressable locations over time; 4) using the signal detected over time and determining an amount of the amplicons in the fluid; and 5) using the amount of the amplicons in the fluid to determine an amount of the nucleotide sequences in the sample. In some embodiments of aspects provided herein, the polymerase-catalyzed chain elongation is a quantitative polymerase chain reaction (Q-PCR), the method further comprises: 1) providing the reaction mixture comprising a nucleic acid sample containing at least one template nucleic acid molecule comprising the target nucleic acid molecule, a primer pair and a polymerase, wherein the primer pair has sequence complementarity with the at least one template nucleic acid molecule, wherein the primer pair comprises a limiting primer and an excess primer, wherein the reaction mixture further comprises at least one of the nucleic acid construct of the present disclosure; 2) subjecting the reaction mixture to the Q-PCR under conditions that are sufficient to yield an amplification product of the template nucleic acid molecule and the limiting primer, which amplicon comprises the limiting primer; 3) bringing the reaction mixture in contact with a sensor array having (i) a substrate comprising a plurality of probes immobilized to a surface of the substrate at different individually addressable locations, wherein the probes have sequence complementarity with the limiting primer and are capable of capturing the limiting primer, and (ii) an array of detectors configured to detect at least one signal from the addressable locations, wherein the at least one signal is indicative of the limiting primer binding with an individual probe of the plurality of probes; 4) using the array of detectors to detect the at least one signal from one or more the addressable locations at multiple time points during the nucleic acid amplification reaction; and 5) detecting the target nucleic acid molecule based on the at least one signal indicative of the limiting primer binding with the individual probe of the plurality of probes.

Aspects of the present disclosure provide a nucleic acid construct, comprising: a) a plurality of nucleotides; and b) one or more photocleavable moieties at 5′-terminus of the nucleic acid construct, wherein the 5′-terminus of the nucleic acid construct is configured to be resistant to cleavage by an exonuclease; wherein each of the one or more photocleavable moieties is independently located: a) on or connected to a nucleobase; b) on or connected to a ribose; or c) a combination thereof.

In some embodiments of aspects provided herein, the nucleic acid construct is configured to form a nucleic acid molecule after photocleavage of the one or more photocleavable moieties, and wherein the nucleic acid molecule is not resistant to the cleavage by the exonuclease. In some embodiments of aspects provided herein, the nucleic acid construct is configured to hybridize to a target nucleic acid molecule and remain resistant to the cleavage by the exonuclease.

Aspects of the present disclosure provide method of conducting a polymerase-catalyzed chain elongation, comprising: a) providing a reaction mixture comprising the nucleic acid construct of the present disclosure, the target nucleic acid molecule, a primer, a polymerase, wherein the target nucleic acid molecule comprises a nucleic acid sequence complimentary to the nucleic acid construct; b) subjecting the reaction mixture to conditions for the polymerase-catalyzed chain elongation of the primer using the target nucleic acid molecule as a template; and c) radiating the reaction mixture or the nucleic acid construct with photons of light; thereby performing the polymerase-catalyzed chain elongation through the nucleic acid sequence.

In some embodiments of aspects provided herein, the subjecting in b) does not enable the performing in c). In some embodiments of aspects provided herein, the nucleic acid construct remains intact in the reaction mixture before the radiating in c). In some embodiments of aspects provided herein, the method further comprises: in c), cleaving the one or more photocleavable moieties. In some embodiments of aspects provided herein, the method further comprises: in c), forming the nucleic acid molecule. In some embodiments of aspects provided herein, the performing in c) comprises digesting the nucleic acid molecule formed in c) after the radiating by the exonuclease, wherein the polymerase is the exonuclease. In some embodiments of aspects provided herein, the performing in c) comprises extending the primer through the nucleic acid sequence after the radiating and/or after the digesting.

Aspects of the present disclosure provide a method of conducting the polymerase-catalyzed chain elongation using the nucleic acid construct, comprising: a) providing a reaction mixture comprising the nucleic acid construct, the template nucleic acid molecule, a polymerase, wherein the nucleic acid construct comprises at least the first sequence located at or near the 3′-terminus and the second sequence located at or near the 5′-terminus, wherein the first sequence is active in the polymerase-catalyzed chain elongation; b) subjecting the reaction mixture to conditions for the polymerase-catalyzed chain elongation, thereby performing the polymerase-catalyzed chain elongation and producing a plurality of first amplicons comprising sequences of both the first sequence and the second sequence or complementary sequence to both the first sequence and the second sequence; and c) radiating the reaction mixture or the nucleic acid construct with photons of light, thereby cleaving the nucleic acid construct, and producing a plurality of second amplicons comprising the first sequence or complementary sequence to the first sequence, with the proviso that each of the plurality of second amplicons does not contain the second sequence or complementary sequence to the second sequence.

Aspects of the present disclosure provide a method of conducting a polymerase-catalyzed chain elongation using at least one of the nucleic acid construct of the present disclosure, comprising: a) providing a reaction mixture comprising the nucleic acid construct, the template nucleic acid molecule, a polymerase; b) subjecting the reaction mixture to conditions for the polymerase-catalyzed chain elongation; and c) radiating the reaction mixture or the nucleic acid construct with photons of light; thereby performing the polymerase-catalyzed chain elongation, wherein the polymerase-catalyzed chain elongation is PCR, RT-PCR, QPCR or qRT-PCR.

In some embodiments of aspects provided herein, the at least one of the nucleic acid construct is a primer for the PCR, RT-PCR, QPCR or qRT-PCR. In some embodiments of aspects provided herein, the at least one of the nucleic acid construct is a solution-phase probe for the PCR, RT-PCR, QPCR or qRT-PCR. In some embodiments of aspects provided herein, the at least one of the nucleic acid construct is an immobilized probe for the PCR, RT-PCR, QPCR or qRT-PCR. In some embodiments of aspects provided herein, the at least one of the nucleic acid construct are more than two nucleic acid constructs, and are a combination of a primer for the PCR, RT-PCR, QPCR or qRT-PCR, a solution-phase probe for the PCR, RT-PCR, QPCR or qRT-PCR, and an immobilized probe for the PCR, RT-PCR, QPCR or qRT-PCR, each of which is independently selected.

Aspects of the present disclosure provide an automated microarray system of quantifying microarray data comprising: a) a solid support having a surface and a plurality of different probes, wherein the plurality of different probes are immobilized to the surface; b) a fluid volume comprising an analyte, wherein the fluid volume is in contact with the solid support, wherein at least one of the plurality of different probes and the analyte comprises at least one of the nucleic acid construct of the present disclosure; c) a detector or a detect assembly configured to detect signals measured at multiple time points from each of a plurality of spots on the solid support while the fluid volume is in contact with the solid support, wherein the signals are optical signals or electrochemical signals; d) a computer configured to convert signals into microarray data, wherein the computer further comprises instructions configured to cause the microarray data to be processed by the computer according to a processing method comprising: 1) determining an estimate of an interaction between the plurality of different probes and the analyte comprising (i) analytical expression and (ii) by calibration of the microarray using at least one standard probe on the solid support; 2) generating a stochastic-matrix that utilizes the estimate in a Markov chain model that comprises modeling hybridization, cross-hybridization, and unbound transition probabilities between states; 3) obtaining affinity-based array data using the detector or the detector assembly; 4) applying the affinity-based array data to the stochastic-matrix; 5) applying an optimization algorithm selected from the group consisting of a maximum likelihood estimation algorithm, a maximum a-posteriori criterion, a constrained least squares calculation, and any combination thereof that exploits and does not suppress non-specific interactions by considering the non-specific interactions as interference rather than noise; and 6) outputting optimized affinity-based array data to a user, wherein the optimized affinity-based array data has an improved signal-to-noise ratio compared to the affinity-based array data obtained by using the detector or the detector assembly.

Aspects of the present disclosure provide an integrated biosensor array, comprising, in order, a molecular recognition layer comprising at least one of the nucleic acid construct of the present disclosure, an optical layer, and a sensor layer integrated in a sandwich configuration, wherein: a) the molecular recognition layer comprises a plurality of different probes attached at different independently addressable locations, each of the independently addressable locations configured to receive an excitation photon flux directly from a single source located on a single side of the molecular recognition layer, wherein the molecular recognition layer transmits light to the optical layer, wherein at one of the plurality of different probes comprises the at least one of the nucleic acid construct; b) the optical layer comprises an optical filter layer, wherein the optical layer transmits light from the molecular recognition layer to the sensor layer, whereby the transmitted light is filtered; and the sensor layer comprises an array of optical sensors that detect the filtered light transmitted through the optical layer, the sensor layer comprising sensor elements fabricated using a CMOS fabrication process; wherein the molecular recognition layer, the optical layer and the sensor layer comprise an integrated structure in which the molecular layer is in contact with the optical layer and the optical layer is in contact with the sensor layer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows examples of photocleavable groups (LG=leaving group);

FIG. 2 shows an example nucleic acid molecule comprising photo-cleavable bonds;

FIG. 3 shows an example of reagents having a photo-cleavable structure;

FIG. 4 shows an example of a nucleic acid construct comprising 3′-end extension inhibitor;

FIG. 5 shows an example of reagents for a 3′-end extension inhibitor;

FIG. 6 shows an example of nucleic acid construct comprising 5′-end exonuclease inhibitor

FIG. 7 shows an example of a 5′-end exonuclease inhibitor;

FIG. 8 shows an example of nucleic acid construct comprising photo-cleavable base-pairing inhibitor;

FIG. 9 shows an example of reagents for a photo-cleavable base-pairing inhibitor;

FIG. 10 shows examples of light-start primers;

FIGS. 11A-11D show examples of light-stop primers;

FIGS. 12A-12B show examples of light-start hybridization probes;

FIG. 13 shows examples of light-stop hybridization probes;

FIG. 14 schematically illustrates an example of 5′-end exonuclease protector

FIG. 15 schematically illustrates an example of light-enabled nested PCR; and

FIG. 16 schematically illustrates another example of light-enabled nested PCR.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides chemically-modified and photo-triggered nucleic acid (NA) constructs that have unique properties, such that the constructs can transform its chemical structure when triggered by photons of light in a photochemical fashion, thereby changing its chemical/biochemical functions. These photo-triggered changing properties of the chemically-modified NA constructs can be utilized in molecular detection reactions/processes.

In some embodiments, the photo-triggered NA constructs can be used in NA detection assays that are used in life-science research and molecular diagnostics. In these assays, NA molecules are the target of the assay and/or are used as molecular recognition elements for the assay. The photo-triggered NA construct is added to the assay such that by appropriately applying photons of light to the system, the photo-triggered NA construct can improve the assay detection accuracy and/or reduce the workflow complexity and/or shorten the turnaround time. Other advantages are also possible.

Some example detection assays are NA amplification tests (NAATs) that use polymerase chain reaction processes; NA affinity-based detection systems that take advantage of 2-dimensional and addressable DNA microarrays; and DNA sequencing arrays that incorporate solid-phase sequence-by-synthesis (SBS) methods.

### Photo-Triggered Nucleic Acid Constructs and Their Use in Operations

The term “photo-triggered nucleic acid construct”, or “NA construct,” as used herein, generally refers to NA molecules that comprise of 1) one or more photosensitive systems or photosensitive chemical moieties that can reside in a first molecular state prior to exposure to photons of light; and 2) one or more DNA or RNA molecules covalently or non-covalently linked to the one or more photosensitive systems or photosensitive chemical moieties. When photons of light are applied to the one or more photosensitive systems or chemical moieties in the nucleic acid construct, the one or more photosensitive systems or photosensitive chemical moieties change from the first molecular state into a second molecular state, which in turn changes the biochemical properties of the NA construct. For example, the photons of light can cause chemical changes in the NA construct by breaking or making chemical bond(s) in the one or more photosensitive systems or photosensitive chemical moieties.

The NA construct can comprise about 1, 2, 3, 4, 5, 6, 7, 8, 9, 10, 20, 30, 40, 50, 60, 70, 80, 90, 100, 200, 300, 400, 500, 600, 700, 800, 900 NA molecules. The NA construct comprises at least 1, 2, 3, 4, 5, 6, 7, 8, 9, 10, 20, 30, 40, 50, 60, 70, 80, 90, 100, 200, 300, 400, 500, 600, 700, 800, 900 NA molecules. The NA construct can comprise no more than 2, 3, 4, 5, 6, 7, 8, 9, 10, 20, 30, 40, 50, 60, 70, 80, 90, 100, 200, 300, 400, 500, 600, 700, 800, 900 NA molecules.

The term, “photosensitive system” or “photosensitive chemical moiety,” as used herein, generally refers to a single or an assortment of chemical structures comprising photo-labile chemical bond(s). The photosensitive system or the photosensitive chemical moiety can absorb wavelength-specific photons to increase the reaction rate of certain chemical reactions in which the photosensitive system or the photosensitive chemical moiety can participate. Other descriptive words, such as light-sensitive, light-cleavable, light-activatable, photolabile, photoactivatable or photocleavable, can be used interchangeably with the word photosensitive.

The term “molecular state” as used herein, generally refers to the atomic and molecular structure and the chemical, physiochemical, biochemical, electrochemical, and photochemical properties that associate with one or more specific molecules, such as, for example, NA constructs.

The term “biochemical properties,” as used herein, generally refers to characteristics of the NA construct in biological and chemical reactions. The biochemical properties of the nucleic acid construct can change depending on the molecular state of the NA construct. The molecular state of the NA construct can change by reactions of the one or more photosensitive systems or photosensitive chemical moieties. In addition, the biochemical properties of the NA construct in the first molecular state can be different from those in the second molecular state.

In some embodiments, the first molecular state is the inactive molecular state for the NA construct while the second molecular state is the active molecular state for the NA construct. In some embodiments, the first molecular state is the active molecular state for the NA construct while the second molecular state is the inactive molecular state for the NA construct.

Each NA construct may have different biochemical property, including different reactivities in biochemical reactions. Examples of biochemical properties can include, for example, whether the NA construct can facilitate, block or participate in a particular biochemical reactions, such as, for example, a polymerase chain reaction or hybridization reaction. The different biochemical properties can be triggered by photons of light.

The biochemical property of a NA construct can include different molecular states of the NA construct. For example, the biochemical properties of the NA construct in the first molecular state can be different from the biochemical properties of the NA construct in the second molecular state. The biochemical properties of the NA construct in the first molecular state and the second molecular state can be designed such that photons of light can start and/or stops specific molecular reactions that the NA construct can participate in. Such changes in molecular state can be triggered by photons of light. Examples of biochemical properties for a primer can be active primers and inactive primer, etc. In some embodiments, the present disclosure describes methods and systems to toggle primers in the extension reactions between “active” and “inactive” molecular states with photons of light. In some embodiments, active/inactive molecular state-switching can be enabled by cleaving a photocleavable bond within a nucleic acid construct. In the present disclosure, the terms of “latent”, “inactivated”, “inert” and “non-functional” are synonymous with the term “inactive”. Similar terminology is used when describing “probes”.

The NA constructs typically reside in a reaction chamber to which photons of light can be applied to by a light source system.

### 1. Photosensitive Systems or Photosensitive Chemical Moieties

A photosensitive system or photosensitive chemical moiety can be a single or a plurality of chemical structures comprising photolabile chemical bond(s). The photosensitive system or photosensitive chemical moiety can change its structure or chemical propertied when radiated by photons of light. The photosensitive system or photosensitive chemical moiety can absorb wavelength-specific photons to increase the reaction rate of certain chemical reactions which the photosensitive system or the photosensitive chemical moiety can facilitate or participate in. For example, these chemical reactions can:
Alter the chemical structures of the photosensitive system or the photosensitive chemical moiety;
Break the structure of the photosensitive system the photosensitive chemical moiety into a plurality of smaller structures;
Add an external chemical structure to the photosensitive system; or
Form an intramolecular bond or bonds within the photosensitive system or the photosensitive chemical moiety;
Form an intermolecular bond or bonds between two or more photosensitive systems or photosensitive chemical moieties or external chemical structures (relative to the photosensitive systems and photosensitive chemical moieties); or
A combination thereof.

In some embodiments, the photosensitive systems or photosensitive chemical moiety can be incorporated within the structure of a nucleic acid molecule. For example, the photosensitive systems or photosensitive chemical moiety can be:
Placed at functional group(s) of the NA, for example, on the hetero atoms of the nucleobase or on the 3′-OH of the ribose ring;
Used as part of a linker group between two NA sequences, wherein, in the presence of photons of light, the linker group can break into smaller groups, thereby separating the two previously linked NA sequences into two independent nucleic acid sequences (i.e., they are not linked any more);
Placed at the 5′-termini of a NA strand, wherein the presence of the photosensitive systems or photosensitive chemical moiety prevents certain biochemical reaction from happening on the 5′-termini of the nucleic acid strand, e.g., a photolabile group on the 5′ phosphate group of the terminal NA;
Placed at the 3′-termini of a NA strand, wherein the presence of the photosensitive systems or photosensitive chemical moiety prevents certain biochemical reaction from happening on the 3′-termini of the NA strand, e.g., a photolabile group on the 3′-OH group of the terminal NA; or
A combination thereof.

Examples of some photosensitive chemical moieties can be found in Mayer, G. and Heckel, A., “Biologically active molecules with a ‘light switch’,” Angew. Chem., Int. Ed., 2006; 45(30), pp.4900-4921, which is entirely incorporated herein by reference. Examples of some photosensitive chemical moieties may include ortho-nitrobenzyloxy linkers, ortho-nitrobenzylamino linkers, alpha-substituted ortho-nitrobenzyl linkers, ortho-nitroveratryl linkers, phenacyl linkers, para-alkoxyphenacyl linkers, benzoin linkers, or pivaloyl linkers. See R.J.T. Mikkelsen, “Photolabile Linkers for Solid-phase Synthesis,” ACS Comb. Sci. 2018; 20(7):377-399; S. Peukert and B. Giese, “The Pivaloylglycol Anchor Group: A New Platform for a Photolabile Linker in Solid-Phase Synthesis,” J. Org. Chem. 1998, 63(24): 9045-9051, each of which is entirely incorporated herein by reference.

For example, nitrobenzyl-based chemical moieties can be, such as, for example, those shown below:

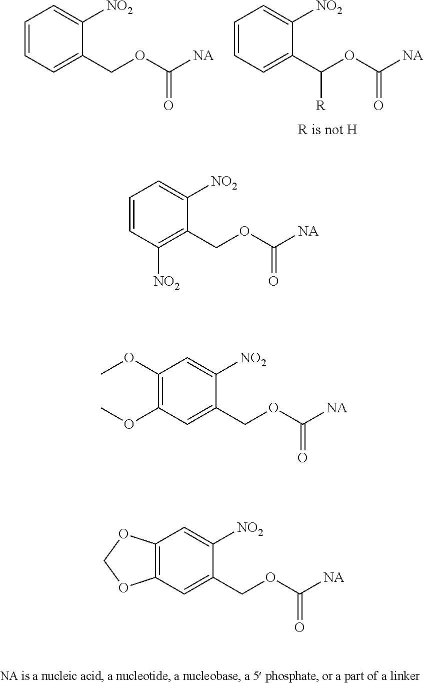

The nitrobenzyl-based chemical moieties may undergo Norrish Type II mechanism with incident photons to provide the cleaved products as shown below:

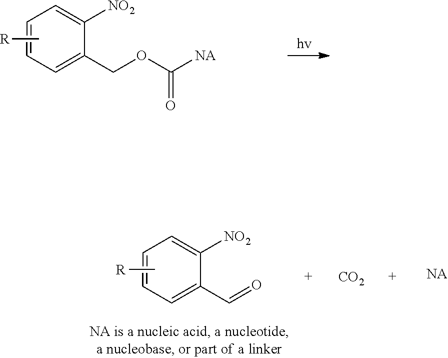

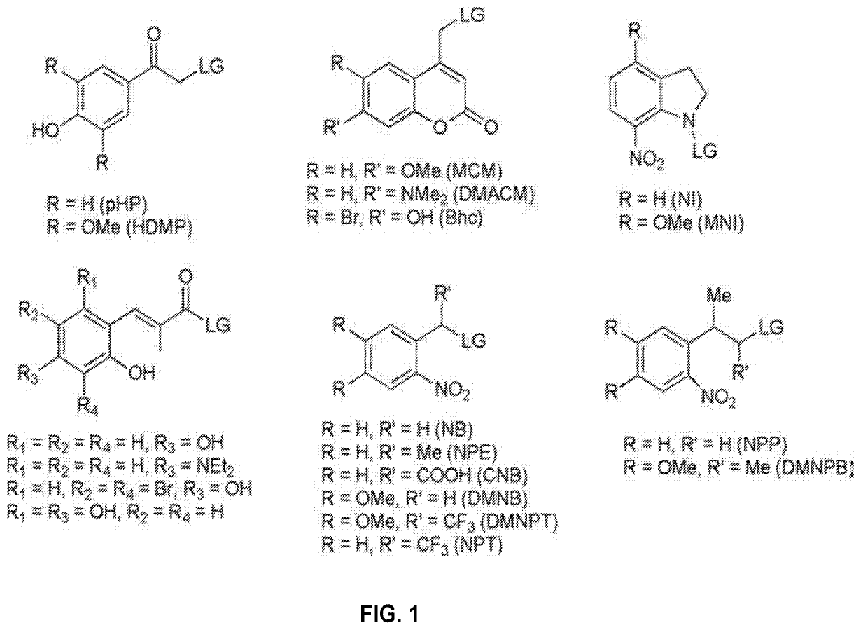

Some examples of photocleavable groups can be found in FIG. 1. LG refers to a leaving group in FIG. 1. Among them, some examples are 4-methoxy-7-nitroindolinyl (MNI), I-nitrobenzyl (O-NB), 3 -(4,5 -dimethoxy-2-nitrophenyl)2-butyl (DMNPB) 4-carboxymethoxy-5,7-dinitroindoinyl (CDNI).

### 2. Molecular States

The term “molecular state” as used herein, generally refers to the atomic and molecular structure and the chemical, physiochemical, biochemical, electrochemical, and photochemical properties that associate with one or more specific molecules, such as, for example, NA constructs. For example, the NA construct can exhibit its molecular state(s) within a defined aqueous environment or under other reaction conditions for nucleic acids in the presence of other molecules. The molecular state of NA constructs may include propensities of the NA constructs to undergo certain reactions, such as, for example, ligations, coupling reactions, chain elongation, chain digestion, etc.

The biochemical property of a NA construct can include different molecular states of the NA construct. For example, the biochemical properties of the NA construct in the first molecular state can be different from the biochemical properties of the NA construct in the second molecular state. The biochemical properties of the NA construct in the first molecular state and the second molecular state can be designed such that photons of light can start and/or stops specific molecular reactions that the NA construct can participate in. Such changes in molecular state can be triggered by photons of light. For example, photochemical reactions can change the molecular structure of a nucleic acid reagent, thereby changing the biochemical properties and reactivities of the nucleic acid reagent in biochemical reactions.

For example, a NA construct can be an “active primer,” which is a primer in the traditional PCR sense that can support nucleotide addition (i.e., extension of the growing strand) facilitated by a polymerase enzyme. In other words, an active primer can be capable of base pairing to a complementary template sequence to form anti-parallel duplex structure at the experimental conditions, and can possess a native (available) 3′-hydroxyl group to which the polymerase enzyme can add another nucleotide, thus extending the primer by at least one base. An “inactive primer” can be a primer that cannot support or facilitate nucleotide addition, either by virtue of its inability to adequately bind the template strand (unable to base pairing) or the absence of an available 3′-hydroxyl group of a terminal nucleotide. For example, placing a photocleavable chemical moiety on the 3′-hydroxyl group of terminal nucleotide can block the polymerase reaction. Upon exposure to light, the photocleavable chemical moiety on the 3′-hydroxyl group can be removed and the resulting free 3′-hydroxyl group can be available for the extension of the growing strand. Similar mechanism can apply in ligase-catalyze reactions in terms of blocking and deblocking the ligation site on the NA. Other examples of base-pairing inhibitors can be chemical groups placed on at least one strand of the DNA (e.g., the growing strand) such that they prevent the DNA strand to bind to its complementary strand due to steric reasons or other chemical reasons.

In some embodiments, the present disclosure describes methods and systems to toggle primers in the extension reactions between “active” and “inactive” molecular states with photons of light. By changing the molecular states of the primers, the present disclosure can enable novel amplification strategies, especially with respect to the “closed tube” methods (i.e., no extra reagents are added after the PCR reaction starts) and “multiplex” methods that are highly desirable in the field of NA amplification-based diagnostics. The present disclosure describes methods to effectively change the composition (and properties, such as, the molecular states) of the primer set during the amplification reaction, without adding or removing reagents or changing the reaction chamber between reactions. Therefore, “inactive” molecular state describes the status and functional state of a particular primer and but not its use. Inactive primers can be made active and vice-versa upon the exposure to the light. Even though the examples below show individual components for simplicity and demonstration, some complex multiplex assays might require up to 10, 20, 30, 40, 50, 60, 70, 80, 90, 100 primers, or even more. The active/inactive molecular state-switching can be triggered by the same light exposure or different light exposure. For example, one photocleavable chemical moiety can react at one wavelength of the light while another photocleavable chemical moiety can react at another wavelength of the light.

In some embodiments, active/inactive molecular state-switching can be enabled by cleaving a photocleavable bond within a NA construct, thereby cutting the original nucleic acid strand(s) into parts. In some embodiments, active/inactive molecular state-switching is enabled by cleaving a photocleavable bond within a NA construct, thereby removing blocking groups from certain nucleic acid units of the NA construct. For example, upon exposure to light, the blocking group on base-pairing inhibitors can be remove and the NA sequence of the NA construct remain intact (i.e., the length and the identities of the sequence of the NA construct remain the same before and after the removal of the blocking groups).

In the present disclosure, the terms of “latent”, “inactivated”, “inert” and “non-functional” are synonymous with the term “inactive”. Similar terminology is used when describing “probes” which are related to signal transduction and would not participate in polymerase-catalyzed extensions such as PCR.

### 3. Biochemical Properties

The term “biochemical properties,” as used herein, generally refers to characteristics of the NA construct in biological and chemical reactions, including, for example, the propensity or ability of the NA construct to engage in certain biochemical or chemical reactions. In addition, the biochemical properties of the NA construct in the first molecular state can be different from those in the second molecular state. One example of such biochemical properties can be the ability of the NA construct to start or stop a molecular reaction after radiation by photons of light. For example, the biochemical properties may include, but are not limited to, the abilities of:
The NA construct in a single stranded form to base-pair with itself and form a hairpin structure, or form a homodimer with another copy of the NA construct, form a heterodimer with another NA molecule;
DNA polymerase enzymes to extend the NA construct using a template NA;
RNA polymerase enzymes to extend the NA construct using a template NA;
Reverse transcriptase enzymes to extend the NA construct using a template NA;
Terminal transferase enzymes to extend the NA construct;
Exonuclease enzymes to digest the NA construct;
Endonuclease enzymes to break the NA construct;
Restriction enzymes to break the NA construct at specific coordinates within its sequence; and
Ligase enzymes to use the NA construct as a substrate or template.

The biochemical properties of the nucleic acid construct can change according to the molecular state of the NA construct. The molecular state of the NA construct can change by reactions of the one or more photosensitive systems or photosensitive chemical moieties.

### 4. Reaction Chamber

The term “reaction chamber,” as used herein, generally refers to a physical system that confines an aqueous solution or other media, and in which the NA constructs resides. The reaction chamber may allow the photons of light to reach the NA constructs residing inside and may have a temperature control to set and dynamically change the temperature within the chamber, such as, the temperature of the aqueous solution.

In some embodiments, the reaction chamber can have a volume ranging from about 0.1 nanoliter (nL) to about 10 milliliter (mL). In some cases, the reaction chamber may have a volume ranging from about 1 microliter (μL) to about 100 μL. In some embodiments, the reaction chamber is about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, or 900 nL. In some embodiments, the reaction chamber is about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, or 900 μL. In some embodiments, the reaction chamber is about 0.1, 0.2, 0.3, 0.4, 0.5, 0.6, 0.7, 0.8, 0.9, 1, 2, 3, 4, 5, 6, 7, 8, 9, or 10 mL.

The reaction chamber can have a temperature ranging from about 4 ° C. to about 100 ° C. The temperature of the reaction chamber can be controlled with accuracies as about ±0.01 ° C., ±0.02 ° C., ±0.03 ° C., ±0.04 ° C., ±0.05 ° C., ±0.06 ° C., ±0.07 ° C., 0.08° C., ±0.09° C., ±0.1° C., ±0.2° C., ±0.3° C., or ±0.4° C. . In some embodiments, the temperature of the reaction chamber may range from about 30 ° C. to about 95 ° C., and the accuracy of controlling the temperature can be controlled to within ±0.1° C.

### 5. Light

The term “light,” as used herein with respect to the reaction chamber, generally refers to the photon flux confined within specific wavelengths and applied to the reaction chamber for a duration of time. The wavelengths of light can be from about 200 nanometer (nm) to about 2000 nm. In some embodiments, the wavelengths of light can from about 200 nm to about 400 nm, from about 300 nm to about 500 nm, or from about 400 nm to about 600 nm. In some embodiments, the total optical power of the light can be from about 0.001 mW/cm² to about 1,000 mW/cm², from about 0.01 mW/cm² to about 100 mW/cm², from about 0.1 mW/cm² to about 10 mW/cm², from about 0.05 mW/cm² to about 20 mW/cm², or from about 0.02 mW/cm² to about 50 mW/cm². The duration of light exposure time can be from about 0.1 second (sec) to about 10,000 sec, from 0.25 sec to about 5,000 sec, from about 0.5 sec to about 1,000 sec, from about 0.75 sec to about 500 sec, from about 1 sec to about 100 sec.

### 6. Light Source

The term “light source system,” as used herein, generally refers to the combination of devices that in concert generate photons of light within defined wavelengths and control its power to be applied to the nucleic acid constructs. The light source system may include a photon source that can be a light-emitting diode (LED), laser source, incandescent lamp, or gas discharge lamp. The light source system may include a power control device to control the light output power. The light source system may include wavelength-selective optical filters to ensure that its output light is within the desired wavelengths. The light source system may include optical devices to focus and/or collimate its output photon flux.

### Modification of Nucleic Acids to Enable Photosensitivity

Various methods can be used to make NA molecules or structures having photochemical properties. For example, a method may comprise the use of solid-support phosphoramidite chemistry. The method may comprise synthesizing or growing nucleic acid sequence on a solid support to a position where a modification may be desired. Next, a special phosphoramidite may be coupled to the growing nucleic acid molecule at the modification position. The modified nucleic acid molecule may or may not be extended after the modification. Once the reaction is completed, the nucleic acid molecule may then be cleaved from the solid support. The cleaved nucleic acid molecule may or may not be subjected to additional reactions or treatment (e.g., purification, modification etc.).

Examples of photosensitive systems or photosensitive chemical moieties, as described above, can be a photocleavable group on part of the nucleotide (either the ribose part or the nucleobase part or between any of the chemical moieties of the nucleic acid), or as a part of a linker between two single stranded nucleic acid. The linker can have two photocleavable bonds, each of which bonds with a nucleic acid segment. There can be many types of modifications of nucleic acids that can enable photosensitivity as shown elsewhere in this disclosure. Below are some specific examples.

### 1. Photo-Cleavable Structures

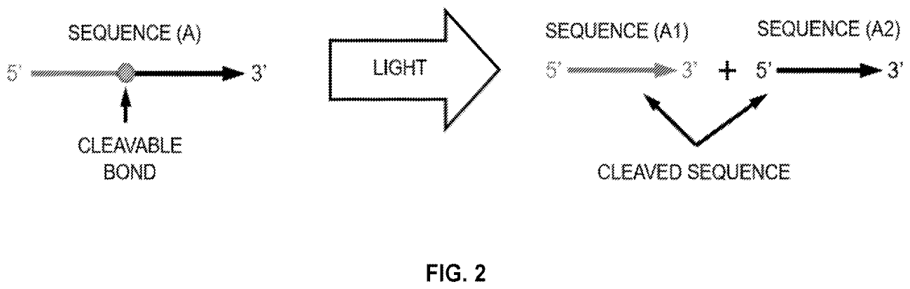

FIG. 2 shows an example NA molecule comprising photo-cleavable bonds. In a photo-cleavable NA structure, two nucleic acid fragments can be linked together by a photosensitive system or photosensitive chemical moiety, which can include one or more photo-cleavable bonds. When the photo-cleavable NA structure molecule is exposed to light from a light source, the molecule may be cleaved into two or more segments due to the present of the photo-cleavable bonds. As a result, the original NA Sequence (A) can be broken into, for example, two smaller pieces of Sequence (Al) and Sequence (A2) as shown in FIG. 2.

In some embodiments, the photosensitive system can be designed such that after the breakage, the cleaved chemical residue remains at the released 3′-end of Sequence (A1) and/or the 5′-end of Sequence (A2). Sequence (A) can be a single stranded or double stranded NA. When Sequence (A) is a double stranded NA, on each strand there may be at least one photo-cleavable bond. In some embodiments, the location of the photo-cleavable bonds may be adjacent to the same pairing NA such that the breakage can produce blunt ends in Sequence (A1) and Sequence (A2), respective. In some embodiment, the location of the photo-cleavable bonds may be staggered on each strand such that after the cleavable, the Sequence (Al) and Sequence (A2) may have sticky ends (overhangs).

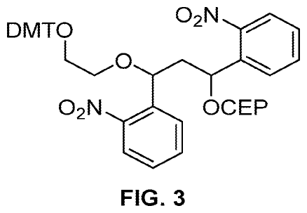

An example compound having photo-cleavable bond(s) is shown in FIG. 3. As shown in FIG. 3 this compound can be used with other DMT phosphoramidite-containing monomers in chemical nucleic acid molecule synthesis of NA construct to insert the photo-cleavable bond(s) into a chain of NA. In the example shown in FIG. 3, the O-nitrobenzyl photolabile blocking groups may link two segments of NA molecules. Without the radiation the photo-labile bonds are intact in the NA construct. The intact NA construct may display molecular state 1 of the biochemical properties of the NA construct. Then upon exposure to a light source the NA construct molecule may be cleaved into two separated nucleic acid fragments, and may yield 3′-hydroxyl and 5′-phosphorylated termini, respectively, in the two newly-formed NA fragments. Due to the breakage of the photo-labile bond(s), the molecular state of the original NA construct may change to new molecular states associated with the two nucleic acid fragments. This is an example of light-triggered molecular state change.

### 2. 3′-End Extension Inhibitors

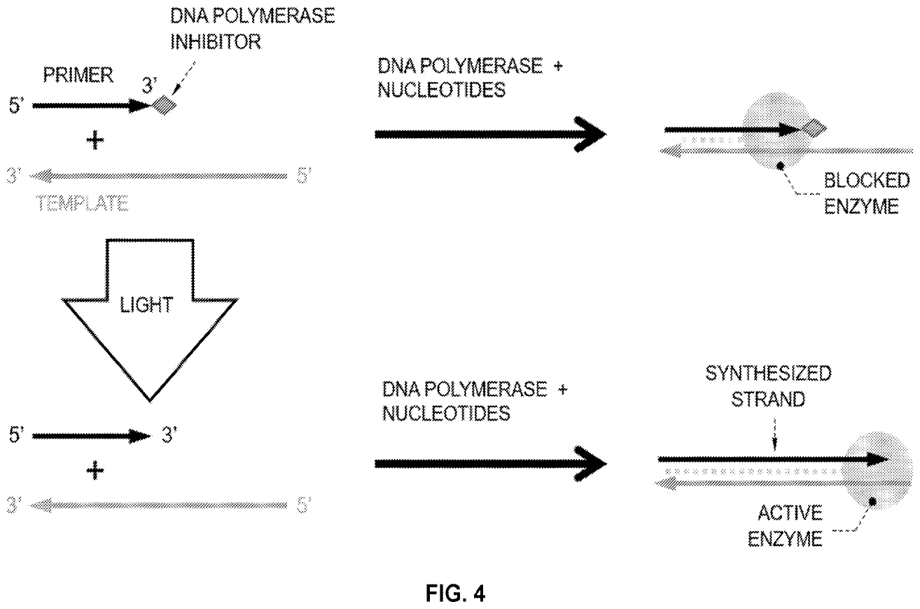

In a NA construct comprising a 3′-end extension inhibitor, a photosensitive system or photosensitive chemical moiety can be chemically attached to the 3′-end terminal unit of the NA sequence. Because of the presence of the 3′-end extension inhibitor, the 3′ extension site is blocked for extension enzymes, including but not limited to, polymerases, transcriptase enzymes, and terminal transferases, etc., so that the enzyme cannot extend the growing strand from the 3′-end terminal unit, and the extension of the growing strand by the enzyme is inhibited. However, exposure to light can remove the blockage and allow the enzymes to extend the growing strand. An example of NA constructs is shown in FIG. 4 wherein the chemical reaction facilitated by a DNA polymerase is initially blocked by the presence of the photosensitive system or photosensitive chemical moiety at the 3′-end of a primer. Then, exposure to light can remove the blocking group and allow the enzyme to synthesize a primed DNA using a template. In this case, a light is directed to the molecule, the polymerase inhibitor may be removed from the molecule, making the molecule extendable by the polymerase.

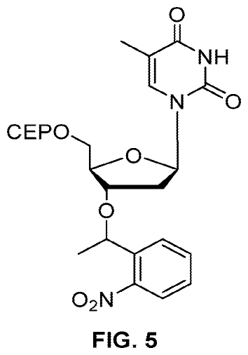

An example of 3′-end terminal unit that can be inserted into a 3′-end polymerase extension inhibitors is shown in FIG. 5. DMT phosphoramidite monomers and this 3′-end terminal unit may be used in the chemical synthesis of nucleic acid molecules (oligonucleotides). Once the 3′-end terminal unit is installed at the 3′-end and in the absence of light exposure, the O-nitrobenzyl photolabile blocking group on the 3′ hydroxy group of the ribose ring of the 3′-end terminal unit may prevent extension at that position by a DNA polymerase. Upon light exposure the blocking group can be removed to reveal the naked 3′ hydroxy group on the ribose ring and restore the extension capability of the NA construct.

### 3. 5′-End Exonuclease Protectors

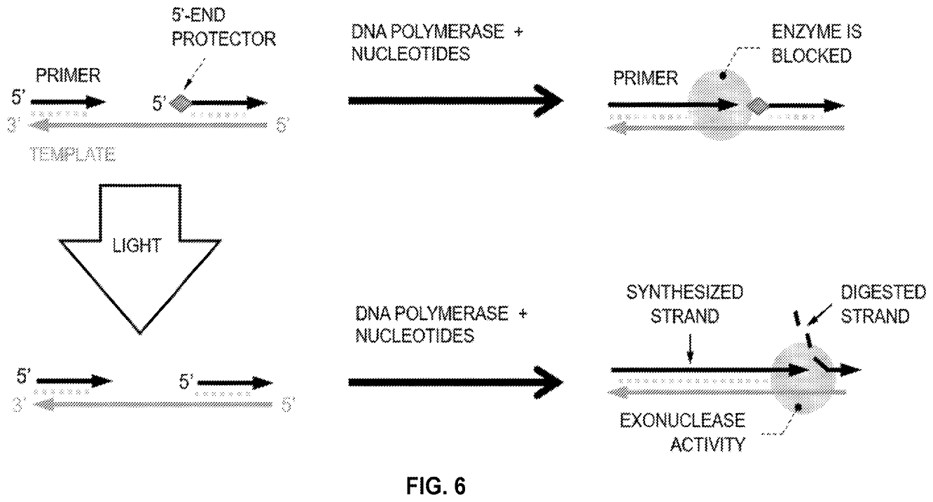

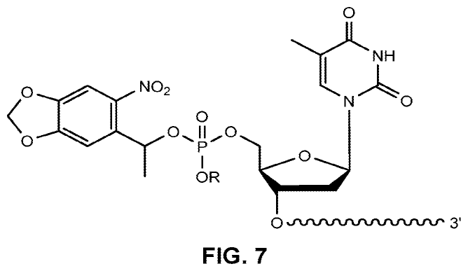

In a NA construct comprising a 5′-end exonuclease protector, a photosensitive system or photosensitive chemical moiety is chemically attached to the 5′-end terminal unit of the nucleic acid sequence. Because of the presence of the 5′-end exonuclease protector, the 5′-end digestion of the strand by exonuclease enzymes can be blocked and the strand is protected from cleavage or digestion. Exposure to light can remove the blockage and allow 5′ to 3′ strand digestion. For example, such a nucleic acid constructs is shown in FIG. 6 where the activities of a DNA polymerase 5′-end exonuclease can be initially blocked, and upon exposure to light and the removal of the 5′-end blocking group, the activities can be regained and allow the enzyme to digest the strand as shown in FIG. 6. Examples of photosensitive system or photosensitive chemical moiety that can be used as 5′-end exonuclease protector is shown in FIG. 7. The hydrophobic tail on the 5′-phosphate diester on the nucleotide can block the digestion of the nucleic acid comprising the nucleotide at the 5′ termini by an exonuclease. Exposure of light on the nucleic acid can remove the blocking group on the 5′-phosphate and allow the digestion by the exonuclease on the 5′ termini nucleotide.

### 4. Base-Pairing Inhibitors

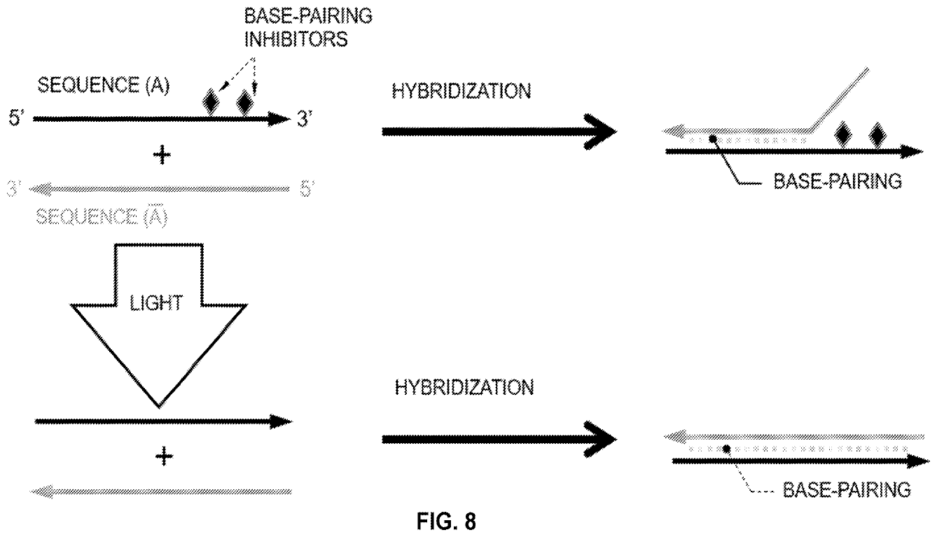

In a NA construct comprising base-pairing inhibitors, a photosensitive system or photosensitive chemical moiety can be chemically attached to one or more nucleobases of the nucleotide units within the NA construct. The base-pairing inhibitors can be in tandem within the nucleic acid sequence or can be distributed within the nucleic acid sequence. The presence of the base-pairing inhibitor can inhibit base-pairing of complementary sequences to the NA construct. Subsequent exposure to light can remove the blocking group and allow the normal base-pairing to occur between the deblocked NA construct and the complementary sequence. An example of such NA constructs is shown in FIG. 8, which shows an example NA molecule comprising photosensitive base-pairing inhibitors. When the NA molecule is not exposed to a light source, due to the existence of base-pair inhibitors, at least a subunit of the NA molecule lacks base-pairing capacity. Such base-pairing capacity may be restored by subjecting the nucleic acid molecule to a light source for a given time period (e.g., greater than or equal to aboutl minute (min), 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, or more). Alternatively, the nucleic acid molecule may be subjected to a light source until the photolysis is complete.

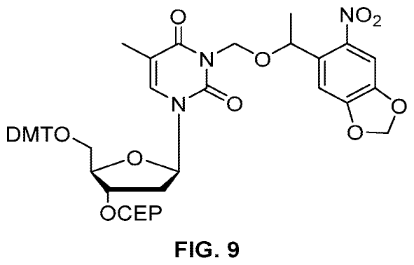

Various compounds can be used as photosensitive base-pairing inhibitors, e.g., a compound as shown in FIG. 9. The reagent shown in FIG. 9 and other DMT phosphoramidite monomers can be used in chemical NA molecule synthesis. Once installed, the O-nitrobenzyl photolabile blocking group may be used to prevent Watson-Crick base pairing due to steric hinderance and/or lack of hydrogen bonding. Upon exposure to a light source (e.g., UV light), the blocking group (shown on the nucleobase) may be removed to restore base pairing capability of the nucleic acid molecules. Photosensitive base-pairing inhibitors that comprising a photo-cleavable chemical moiety on the nucleobase, such as, for example, the compound shown in FIG. 9 (or other similar compounds that have different nucleobases with the photocleavable chemical moiety attached to a hetero atom such as nitrogen or oxygen on the nucleobase) can be made according to H. Lusic, et al., “Photochemical DNA Activation,” Org. Lett., 2007, 9(10): 1903-1906; U.S. PG. Pub. No. 2010/0099159; each of which is entirely incorporated herein by reference.

The different chemical modifications on a nucleic acid, as disclosed above, can be used to build different types of NA constructs, as shown below, for different utilizations.

### Types of Photo-Triggered Nucleic Acid Constructs and Uses Thereof

Also provided herein are NA constructs which have unique biochemical properties relevant to molecular detection that may be triggered when the NA molecules are exposed to a photons of light. These NA constructs, while being used in a reaction chamber, may enhance or decrease the rate, specificity, yield and/or fidelity of the biochemical reaction that are used in common molecular detection assays. Example reactions are polymerase chain reaction (PCR), polymerase-catalyzed chain elongation, reverse transcription polymerase chain reaction (RT-PCR), ligation, terminal transferases extension, hybridization, exonuclease digest, endonuclease digest, and restriction digest, among others. If a reaction comprises of NA components functioning as the target and/or reagent and/or catalyst and/or others, the present disclosure can be used to moderate the reaction by replacing the native component with NA constructs or inserting NA construct into the native components. Examples of nucleic acid molecules or structures having photochemical properties may include, but not limited to, primers, oligonucleotides, polynucleotides, oligonucleotide-containing molecules, nucleotides, or nucleic acid probes. The nucleic acid probes may include hybridization probes which may selectively interact with a target analytes (such as amplicons) during or at the end of a given reaction (such as PCR or RT-PCR). There can be many different types of nucleic acid constructs as shown below.

### 1. Light-Start Primers

Light-start primers are NA sequences that cannot base-pair with a complementary NA sequence template and/or cannot create an initiation site for nucleic acid synthesis enzymes due to the presence of the photosensitive systems or photosensitive chemical moieties, or blocking groups comprising or connected to the photosensitive systems or photosensitive chemical moieties. When light is applied, these light-start primers can remove the blocking group(s) and subsequently become enabled for nucleic acid synthesis in the presence of a nucleic acid template and a nucleic acid synthesis enzyme.

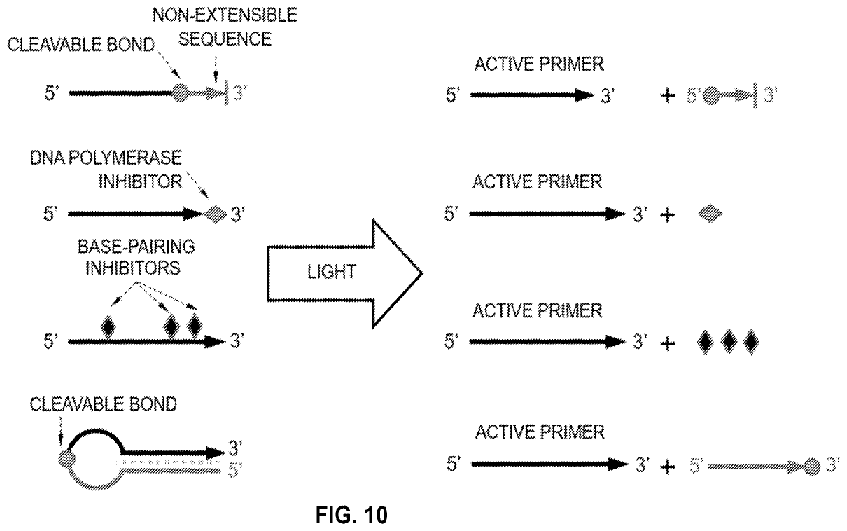

FIG. 10 shows examples of light-start primers and their applications in biochemical processes with their corresponding example sequences listed in Table I. In one embodiment, the primers may comprise an internal photo-cleavable bond modification in a linear NA construct (e.g., a primer), wherein the light-start primer is designed with a photo-cleavable modification such that upon exposure to the light the blocking strand can be removed, and the resulting primer can form a proper initiation site for the polymerase to act on (FIG. 10, top panel). In another embodiment, the primers may comprise a polymerase blocker at the 3′ terminus of a nucleic acid construct (e.g., a primer), wherein the light-start primer is designed using a 3′-end extension inhibitor modification such that when applying light the inhibition can be removed, thereby creating a proper initiation site for the polymerase to act on (FIG. 10, second panel from the top). In one embodiment, the primers may comprise one or more base-pairing inhibitors distributed within the sequence of a NA construct (e.g., a primer), wherein the light-start primer is designed using a base-pairing inhibitor modification such that, initially, the primer comprising the base-pair inhibitors cannot hybridize to the template or form the initiation site for the polymerase. When upon exposure to the light, the inhibition can be removed, and the resulting primer can create a proper initiation site for the polymerase to act on (FIG. 10, third panel from the top). In another embodiment, the primers may comprise a cleavable bond within a hairpin structure of a nucleic acid (e.g., a hairpin primer) the light-start primer is designed using a photo-cleavable hairpin monomer structure. Initially, the 3′-end region of the primer can be unavailable for base-pairing due to the presence of the hairpin. When exposure to the light, the hairpin can be destroyed, and the resulting primer can become available for extension (FIG. 10, bottom panel). In some embodiments, the primers may not be active (i.e., in the inactive molecular state) prior to being exposed to a light source due to the presence of the photosensitive systems or photosensitive chemical moieties on the nucleic acid constructs. However, when the light-start primers are subjected to a light source, the light from the light source may remove some or all of the inhibitors/blockers, or cleave the cleavable bonds comprised in the light-start primers, thereby restoring the capability of the primers into the active molecular state.

TABLE I
Example sequences for light-start primers.

| SEQ ID | | |
|---|---|---|
| NO | Sequence | Primer Type |
| 1 | 5′-CTCGGTCGTCCAATATCGAA[PC]AACT- | internal cleavable bond |
| | 3′-[EI] | modification |
| 2 | 5′-CTCGGTCGTCCAATATCGAA-3′- | 3′-end extension inhibitor |
| | [PCEI] | modification |
| 3 | 5′-CTCGG [symbol] CGT [symbol] AATATCG [symbol] -3′ | base-pairing inhibitors |
| 4 | 5′-TTCGATATT[PC]CTCGGTCGTCC | a cleavable bond within a |
| | AATATCGAA-3′ | hairpin |

[PC]: Photo-cleavable modification
[EI]: Extension inhibitor
[PCEI]: Photo-cleavable polymerase extension inhibitor [symbol]
: Nucleobases with photo-cleavable/photo-removable base pairing inhibitors

### 2. Light-Stop Primers

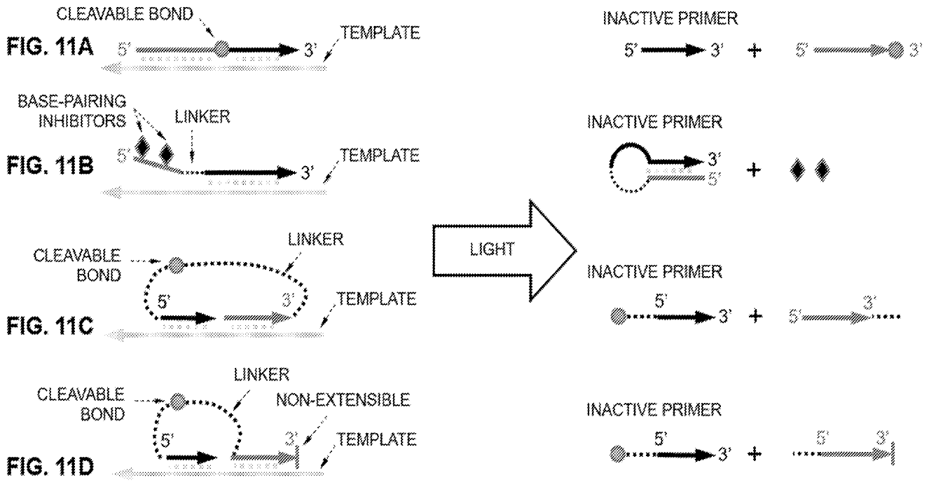

Light-stop primers are NA constructs that can act as the initiation site for polymerases and facilitate NA synthesis in the presence of a nucleic acid template. When light is applied, these light-stop primers can become inactive and cannot enable further NA synthesis. The light-stop primers may be active prior to a light exposure, but may become inactive after being subjected to a light source. FIGS. 11A-11D demonstrate examples of light-stop primers and applications thereof with their corresponding example sequences listed in Table II.

FIGS. 11A and 11B show examples of the light-stop cooperative primers comprising photo-cleavable modifications. Applying light can break the NA constructs and subsequently make the priming thermodynamically unfavorable. The primers may comprise a cleavable bond between two segments of the primer and may require the two linked segments of the primer to hybridize to the same template in order to form a stable primer-template heterodimer. The primers may be active to enzymatic reactions prior to a light exposure. After exposed to the light, the two segments of the cooperative primer may be separated due to the cleavage of the cleavable bond, and may become inactive because the binding of only one segment to the template may become thermodynamically unfavorable for the primer-template heterodimer for each segment.

FIGS. 11C and 11D show examples of light-stop hybridization primers. In FIG. 11C the light-stop primer is designed with a photo-cleavable modification such that applying light can break the primer into separated parts, and can subsequently reduce the base-pairing strength of the transformed primer-template heterodimer, As a result, the priming becomes thermodynamically unfavorable and the primer-template heterodimer can be broken. In FIG. 11D the light-stop primer is designed using a base-pairing inhibitor modifications distributed in the sequence of the primer. Applying light can remove the inhibition and can subsequently create a stable hairpin structure for the transformed primer. Because the transformed primer forms a hairpin structure, the primer-template heterodimer can be disrupted since it is thermodynamically unfavorable for the intermolecular hybridization when compared with the intramolecular hybridization of the hairpin structure.

TABLE II
Example sequences for light-stop primers.

| SEQ ID NO | Sequence | Primer Type |
|---|---|---|
| 5 | 5′-CTCGGTCGTCCA[PC]ATATCGAA- | internal photo-cleavable bond |
| | 3′ | modification |
| 6 | 5′- [symbol] T [symbol] A [symbol] ATT[LK]CTCGGTC | base-pairing inhibitors |
| | GTCCAATATCGAA-3′ | |
| 7 | 5′-CGTCCAATATCGAA- | co-operative primer systems with |
| | 3′[LK][PC][LK]5′-CTCGGT-3′ | photo-cleavable modification |
| 8 | 3′-CTGGCTC-5′[LK][PC][LK]5′- | co-operative primer systems with |
| | GTCCAATATCGAA-3′ | photo-cleavable modification |

[LK]: Non-extensible linker
[PC]: Photo-cleavable modification [symbol]
: Nucleobases with photo-cleavable/photo-removable base pairing inhibitors

### 3. Light-Start Hybridization Probes

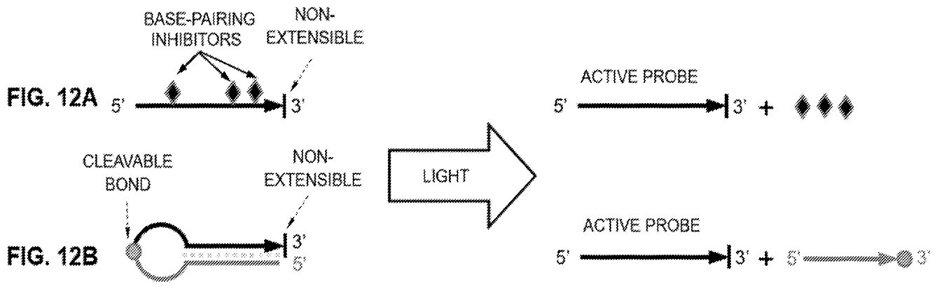

Light-start hybridization probes are NA constructs that can specifically identify with and base pair with their complementary sequence only after light is applied. Prior to that, the light-start hybridization probes are inactive and cannot hybridize to their complementary sequence. Examples of light-start hybridization probes are shown in FIGS. 12A and 12B with their corresponding example sequences listed in Table III.

In FIG. 12A the light-start hybridization probe is designed using a base-pairing inhibitor modification. Initially, the light-start hybridization probe comprising the base-pair inhibitors cannot hybridize to its complementary sequence. Applying light can remove the inhibition to hybridization due to the removal of the base-pairing inhibitors and can allow base-pairing, thereby the transformed light-start hybridization probe can hybridize to its complementary sequence.

In FIG. 12B the light-start hybridization probe is designed to comprise a photo-cleavable hairpin monomer structure. Initially, the probe base-pairing to its complementary sequence is thermodynamically unfavorable due to the presence of the hairpin monomer having intramolecular hybridization. Applying light can disrupt the hairpin by cutting the probe into two separated parts, and make the transformed probe available for base-pairing and hybridization to its complementary sequence.

TABLE III
Example sequences for light-start hybridization probes.

| SEQ ID | | Hybridization Probe |
|---|---|---|
| NO | Sequence | Type |
| 9 | 5′-AC [symbol] TTA [symbol] GAT [symbol] C-3'-[EI] | base-pairing inhibitors |
| 10 | 5′-GCATCCTAACGGTTAA[PC]AATAC | Hairpins with photo- |
| | CGTTAGGATGC-3′-[EI] | cleavable modification |

[EI]: Extension inhibitor
[PC]: Photo-cleavable modification [symbol]
: Nucleobases with photo-cleavable/photo-removable base pairing inhibitors

### 4. Light-Stop Hybridization Probes

Light-stop hybridization probes are nucleic acid constructs that can specifically identify with and base pair with their complementary sequence. However, upon exposure to light, they can become inactive and cannot hybridize to their complementary sequence anymore.

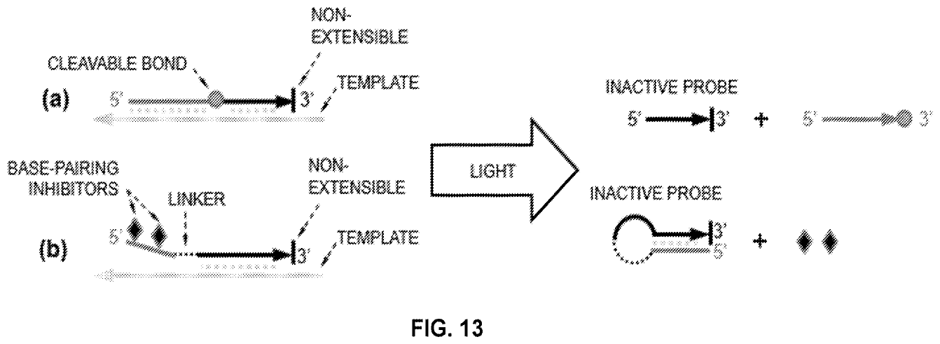

In FIG. 13 examples are shown for the light-stop hybridization probe structures with their corresponding example sequences listed in Table IV.

In FIG. 13, top panel, a the light-stop hybridization probe is designed to comprise a photo-cleavable modification linking two segments of the light-stop hybridization probe. Before exposure to light, both segments of the light-stop hybridization probe hybridize to the target NA, thereby staying in the active molecular state. Upon exposure to light, the light-stop hybridization probe can break the photo-cleavable bond, thereby producing two unlinked segments of the light-stop nucleic acid probe, and can make the probe-template heterodimer formation thermodynamically unfavorable. For example, at least one segment can be designed to have non-complementary sequences with respect to the target nucleic acid and may provide a signal change if not hybridized with the target nucleic acid or separate from the other segment of the light-stop hybridization probe. At least one segment of the transformed light-stop hybridization probe can be in the inactive molecular state.

In FIG. 13, bottom panel, the light-stop hybridization probe is designed to comprise one or more base-pairing inhibitors. Before exposure to light, the presence of the base-pairing inhibitors may prevent self-base pairing within the light-stop hybridization probe to form a hairpin structure. Instead, the light-stop hybridization probe hybridize to the target nucleic acid, thereby staying in the active molecular state. Upon exposure to light, the light-stop hybridization probe can remove the base-pairing inhibition and can create a stable hairpin structure for at least one segment of the transformed light-stop hybridization probe, thereby making the probe-template heterodimer formation thermodynamically unfavorable. The hairpin structure of the light-stop hybridization probe can be in the inactive molecular state.

TABLE IV
Example sequences for light-stop hybridization probes.

| SEQ | | Hybridization Probe |
|---|---|---|
| ID NO | Sequence | Type |
| 11 | 5′-ACCGTTA[PC]GGATGC-3′-[EI] | photo-cleavable |
| | | modification |
| 12 | 5′-G [symbol] TCC [symbol] AAC [symbol] T[LK]AATACC | base-pairing inhibitors |
| | GTTAGGATGC-3′-[EI] | |

[EI]: Extension inhibitor
[PC]: Photo-cleavable modification [symbol]
: Nucleobases with photo-cleavable/photo-removable base pairing inhibitors

### 5. Light-Start 5′-End Exonuclease Probes

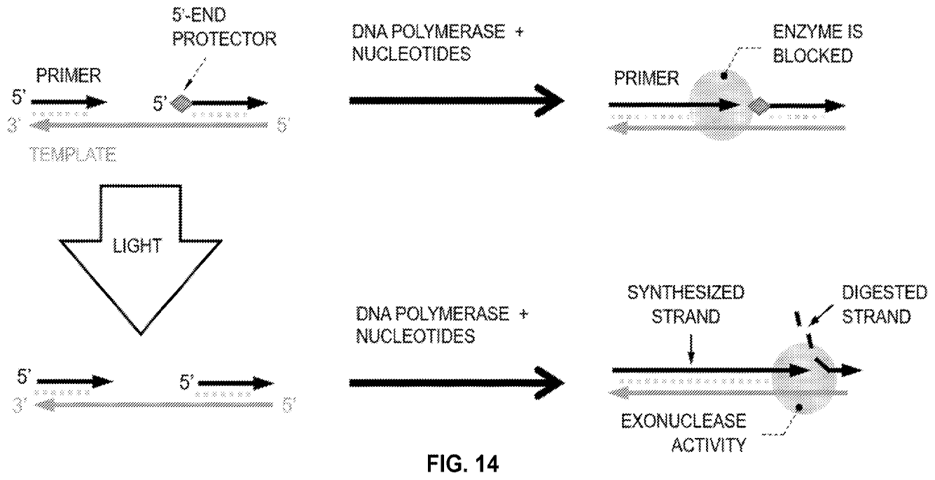

Light-start 5′-end exonuclease probes are NA constructs comprising a 5′-end exonuclease protector modification that can be removed by light. The 5′-end exonuclease protector can be a photosensitive system or photosensitive chemical moiety chemically attached to the 5′-end terminal unit of the NA sequence. Because of the presence of the 5′-end exonuclease protector, the 5′-end digestion of the nucleic acid strand by exonuclease enzymes can be blocked and the nucleic acid strand is protected from cleavage or digestion. The light-start 5′-end exonuclease probes are in the inactive molecular state. Upon exposure to light the 5′-end exonuclease protector can be removed, and 5′ to 3′ strand digestion can be facilitated. For example, such a nucleic acid constructs is shown in FIG. 14 where DNA polymerase 5′-end exonuclease is initially blocked, and exposure to light can remove the blocking group and allow the enzyme to digest the strand.

In general, heteroatoms on the nucleobase, 3′-OH, 5′-OH, and the phosphate group (at either 3′ or 5′ positions) can bond to a photosensitive chemical moiety, such as, for example, any one shown in FIG. 1. A photocleavable linker can have one or more photosensitive chemical moieties attached to the ends of the linker such that upon exposure to light, the one or more photosensitive chemical moieties can break away from the nucleic acid fragments they attached to. Various photocleavable chemical moieties can be used in various ways.

### Example Embodiments with Photo-Triggered NA Constructs

### EXAMPLE 1

### Light-Start PCR

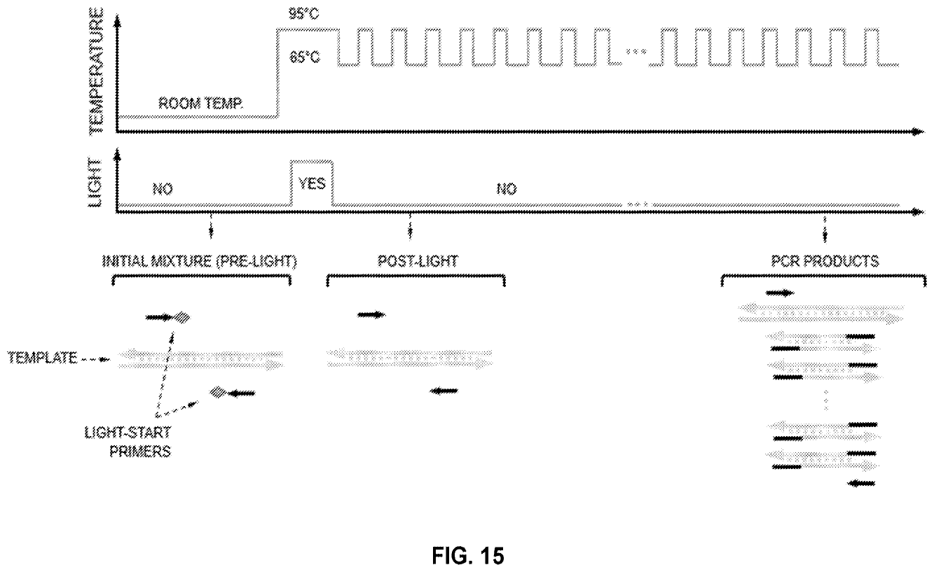

In this example, as depicted in FIG. 15, light-start primer pairs are used in a PCR assay. As depicted in FIG. 15, the PCR and elongation of primers starts after light is applied, but cannot start before the light is applied. Prior to the exposure to the light, neither polymerization, nor exponential amplification can occur because the primers are inactive due to the presence of 3′-end extension inhibitors (i.e., polymerase inhibitors at the 3′-end of the primers). The advantage of this method can be that it may reduce the presence of undesired products and/or primer-dimers that are due to non-specific DNA amplification at room (or colder) temperatures, for example during the introduction of the sample to the reaction or other pre-processing steps. Upon exposure to the light, the 3′-end extension inhibitors can be removed, and the primers can become active in polymerase-catalyzed extensions (i.e., extension of the growing strand, elongation).

This method, which henceforth can be referred to as “light-start PCR”, can be an alternative to other PCR methods, such as, for example, hot-start PCR methods, where heating at elevated temperatures activate the amplification process. Sharkey D J, Scalice E R, Christy K G, Atwood S M, Daiss J L, “Antibodies as thermolabile switches: high temperature triggering for the polymerase chain reaction See Bio/Technology,” 1994, 12(5): 506-9; N. Paul, J. Shum, T. Le, “Hot start PCR,” Methods in Molecular Biology, Humana Press, 2010, 630: 301-18. Thus, light-start PCR may not include reagents and molecules that act as thermolabile switches.

In some embodiments of this invention, both light-start PCR and hot-start PCR methods can be used to better ensure that the amplification remains inactive at lower temperatures and prior to PCR.

In some embodiments of this invention the light-start PCR is included in a quantitative PCR (Q-PCR) system. In some embodiments, a method employing the light-start PCR is a Q-PCR method comprising; (a) performing a nucleic acid amplification on two or more nucleotide sequences in the presence of at one light-start primer to produce two or more amplicons in a fluid; (b) providing an array comprising a solid surface with a plurality of nucleic acid probes at independently addressable locations, said array configured to contact said fluid; and (c) measuring the hybridization of the amplicons to the two or more nucleic acid probes while the fluid is in contact with the array to obtain an amplicon hybridization measurement wherein the amplicons comprise a quencher. In some embodiments, the primers comprising the light-primer are used to create the amplicons and the primers comprise a quencher. In some embodiments, one of the primers in a primer pair comprises a quencher. In some embodiments, both the primers in a primer pair comprise a quencher. In some embodiments, the quenchers are incorporated into the amplicons as they are formed. In some embodiments, deoxynucleotide triphosphates (d-NTP's) are used to make the amplicons, and one or more of the d-NTP's used to make the amplicon comprises a quencher. In some embodiments, the amplicon hybridization measurement is performed by measuring fluorescence from fluorescent moieties attached to the solid surface. In some embodiments, the fluorescent moieties are covalently attached to the nucleic acid probes. In some embodiments, the fluorescent moieties are attached to the substrate and are not covalently attached to the nucleic acid probes. In some embodiments, the amplicons comprise quenchers, and the measuring of hybridization is performed by measuring a decrease in fluorescence due to hybridization of amplicons to the nucleic acid probes.

In some embodiments, a method employing the light-start PCR is a Q-PCR method comprising: (a) providing an array comprising a solid support having a surface and a plurality of different probes, the different probes immobilized to the surface at different addressable locations, each addressable location comprising a fluorescent moiety; (b) performing PCR amplification on a sample comprising a plurality of nucleotide sequences; the PCR amplification carried out in a fluid, wherein:(i) a PCR primer for each nucleic acid sequence is a light-start primer and comprises a quencher; and(ii) the fluid is in contact with the probes, whereby amplified molecules can hybridize with probes, thereby quenching signal from the fluorescent moiety; (c) detecting the signals from the fluorescent moieties at the addressable locations over time; (d) using the signals detected over time to determine the amount of amplified molecules in the fluid; and (e) using the amount of amplified molecules in the fluid to determine the amount of the nucleotide sequences in the sample. In some embodiments, the determining of the amount of amplified molecules is performed during or after multiple temperature cycles of the PCR amplification. In some embodiments, more than one PCR primer for each nucleic acid sequence comprises a quencher. In some embodiments, the detecting of the signals from the fluorescent moieties at the addressable locations over time comprises measuring the rate of hybridization of the amplified molecules with the probes. In some embodiments, the sample comprises messenger RNA or nucleotide sequences derived from messenger RNA, and the determination of the amount of nucleic acid sequence in the sample is used to determine the level of gene expression in a cell or group of cells from which the sample was derived. In some embodiments, the sample comprises genomic DNA or nucleotide sequences derived from genomic DNA, and the determination of the amount of nucleic acid sequence in the sample is used to determine the genetic makeup of a cell or group of cells from which the sample was derived. In some embodiments, two or more PCR primers corresponding to two or more different nucleotide sequences have different quenchers. In some embodiments, two or more different addressable locations comprise different fluorescent moieties. In some embodiments, the different quenchers and/or different fluorescent moieties are used to determine cross-hybridization. In some embodiments, a diagnostic test for determining the state of health of an individual comprising performing the method of performing the Q-PCR method using a light-start primer on a sample from such individual.

In some embodiments, the Q-PCR method is a method for assaying at least one target nucleic acid molecule, comprising: (a) providing a reaction mixture comprising a nucleic acid sample containing at least one template nucleic acid molecule, a primer pair comprising said light-start primer and a polymerase, wherein the primer pair has sequence complementarity with the template nucleic acid molecule, and wherein the primer pair comprises a limiting primer and an excess primer; (b) subjecting the reaction mixture to a nucleic acid amplification reaction under conditions that are sufficient to yield the at least one target nucleic acid molecule as an amplification product of the template nucleic acid molecule and the limiting primer, which at least one target nucleic acid molecule comprises the limiting primer; (c) bringing the reaction mixture in contact with a sensor array having (i) a substrate comprising a plurality of probes immobilized to a surface of the substrate at different individually addressable locations, wherein the probes have sequence complementarity with the limiting primer and are capable of capturing the limiting primer, and (ii) an array of detectors configured to detect at least one signal from the addressable locations, wherein the at least one signal is indicative of the limiting primer binding with an individual probe of the plurality of probes; (d) using the array of detectors to detect the at least one signal from one or more the addressable locations at multiple time points during the nucleic acid amplification reaction; and (e) detecting the target nucleic acid molecule based on the at least one signal indicative of the limiting primer binding with the individual probe of the plurality of probes. In some embodiments, the at least one signal is produced upon binding of the probes to the limiting primer. In some embodiments, the reaction mixture comprises a plurality of limiting primers having different nucleic acid sequences, and the probes specifically bind to the plurality of the limiting primers. In some embodiments, the reaction mixture is provided in a reaction chamber configured to retain the reaction mixture and permit the probes to bind to the limiting primer. In some embodiments, the method further comprises correlating the detected at least one signal at multiple time points with an original concentration of the at least one template nucleic acid molecule by analyzing a binding rate of the probes with the limiting primer. In some embodiments, the probes are oligonucleotides. In some embodiments, the target nucleic acid molecule forms a hairpin loop when hybridized to an individual probe. In some embodiments, the sensor array comprises at least about 100 integrated sensors. In some embodiments, the at least one signal is an optical signal that is indicative of an interaction between an energy acceptor and an energy donor. In some embodiments, the energy acceptor is coupled to the excess primer and/or the limiting primer. In some embodiments, the energy acceptor is coupled to the target nucleic acid molecule. In some embodiments, the energy acceptor is a quencher. In some embodiments, the energy donor is a fluorophore. In some embodiments, the at least one signal is an electrical signal that is indicative of an interaction between an electrode and a redox label. In some embodiments, the redox label is coupled to the excess primer and/or the limiting primer. In some embodiments, the redox label is coupled to the target nucleic acid molecule. In some embodiments, (d) comprises measuring an increase in the at least one signal relative to background. In some embodiments, (d) comprises measuring a decrease in the at least one signal relative to background. In some embodiments, the target nucleic acid molecule is detected at a sensitivity of at least about 90%. In some embodiments, the at least one signal is detected while the reaction mixture comprising the target nucleic acid molecule is in fluid contact with the sensor array. In some embodiments, (b) comprises generating a plurality of target nucleic acid molecules having sequence complementarity with the template nucleic acid. In some embodiments, the array of detectors is configured to detect a plurality of signals from the addressable locations, wherein each of the plurality of signals is indicative of the limiting primer binding with an individual probe of the plurality of probes. In some embodiments, (d) comprises using the array of detectors to detect a plurality of signals from the addressable locations at the multiple time points, wherein each of the plurality of signals is indicative of the limiting primer binding with an individual probe of the plurality of probes. In some embodiments, (e) comprises identifying the limiting primer.

In some embodiments, the present disclosure provides a system for assaying at least one target nucleic acid molecule, comprising: (a) a reaction chamber comprising a reaction mixture comprising a nucleic acid sample containing at least one template nucleic acid molecule, a primer pair that has sequence complementary to the template nucleic acid molecule, and a polymerase, wherein the primer pair comprises a limiting primer and an excess primer, wherein the reaction chamber comprising the reaction mixture is configured to facilitate a nucleic acid amplification reaction on the reaction mixture to yield at least one target nucleic acid molecule as an amplification product of the template nucleic acid; (b) a sensor array comprising (i) a substrate comprising a plurality of probes immobilized to a surface of the substrate at different individually addressable locations, wherein the probes have sequence complementarity with the limiting primer and are capable of capturing the limiting primer; and (ii) an array of detectors configured to detect at least one signal from the addressable locations, wherein the at least one signal is indicative of the limiting primer binding with an individual probe of the plurality of probes; and (c) a computer processor coupled to the sensor array and programmed to (i) subject the reaction mixture to the nucleic acid amplification reaction, and (ii) detect the at least one signal from one or more of the addressable locations at multiple time points during the nucleic acid amplification reaction.

In some embodiments, the Q-PCR method is a method for assaying at least one template nucleic acid molecule, comprising: (a) activating a sensor array comprising (i) a substrate comprising a plurality of first probes immobilized to a first pixel, a plurality of second probes immobilized to a second pixel, wherein the first probes are configured to capture an individual primer of a primer set, and wherein the second probes are configured to capture a control nucleic acid molecule, and (ii) an array of detectors configured to detect at least one first signal from the first pixel and at least one second signal from the second pixel, wherein a difference between the at least one first signal and the at least one second signal over time is indicative of the individual primer binding with an individual probe of the plurality of first probes; (b) subjecting a reaction mixture to a nucleic acid amplification reaction under conditions sufficient to yield at least one target nucleic acid molecule as an amplification product(s) of the template nucleic acid molecule, wherein the reaction mixture comprises (i) a nucleic acid sample containing or suspected of containing the template nucleic acid molecule, (ii) the primer set, (iii) the control nucleic acid molecule, and (iv) a polymerizing enzyme, wherein the individual primer of the primer set has sequence complementarity with the template nucleic acid molecule; (c) using the array of detectors to detect the at least one first signal and the at least one second signal at multiple time points during the nucleic acid amplification reaction; and (d) using the difference between the at least one first signal and the at least one second signal to detect the template nucleic acid molecule. In some embodiments, the at least one first signal is produced upon binding of the individual probe to the individual primer, and wherein the at least one second signal is produced upon binding of an additional probe of the second probes to the control nucleic acid molecule. In some embodiments, the control nucleic acid molecule is not amplified in the amplification reaction. In some embodiments, the reaction mixture comprises a plurality of template nucleic acid molecules, and wherein the first probes specifically bind to a plurality of target nucleic molecules as amplification products of the plurality of the template nucleic acid molecules. In some embodiments, the primer set comprises a plurality of individual primers having different nucleic acid sequences, and wherein the first probes are configured to specifically bind to the plurality of the individual primers. In some embodiments, the reaction mixture is provided in a reaction chamber configured to retain the reaction mixture and permit the first and second probes to bind to the individual primer and the control nucleic acid molecule. In some embodiments, the method further comprises correlating the at least one first signal detected at multiple time points with an initial concentration of the at least one template nucleic acid molecule by analyzing a binding rate of the probes with the individual primer from the primer set. In some embodiments, the first probes or the second probes are oligonucleotides. In some embodiments, the sensor array comprises at least about 100 integrated sensors. In some embodiments, the at least one first signal is a first optical signal that is indicative of a first interaction between a first energy acceptor and a first energy donor associated with the individual primer and the individual probe, and wherein the at least one second signal is a second optical signal that is indicative of a second interaction between a second energy acceptor and a second energy donor associated with the control nucleic acid molecule and an additional probe of the second probes. In some embodiments, the first energy acceptor is coupled to the individual primer, and wherein the second energy acceptor is coupled to the control nucleic acid molecule. In some embodiments, the first energy acceptor is coupled to the target nucleic acid molecule. In some embodiments, the first energy acceptor is a first quencher, and wherein the second energy acceptor is a second quencher. In some embodiments, the first energy donor is a first fluorophore, and wherein the second energy donor is a second fluorophore. In some embodiments, the first energy donor is coupled to the first probe, and wherein the second energy donor is coupled to the second probe. In some embodiments, the target nucleic acid molecule is detected at a sensitivity of at least about 90%. In some embodiments, the at least one first signal is detected while the reaction mixture comprising the target nucleic acid molecule is in fluid contact with the sensor array.

In some embodiments, the Q-PCR system is for assaying at least one template nucleic acid molecule, comprising: (a) a reaction chamber comprising a reaction mixture, wherein the reaction mixture comprises (i) a nucleic acid sample containing or suspected of containing the template nucleic acid molecule, (ii) a primer set comprising an individual primer, (iii) a control nucleic acid molecule, and (iv) a polymerizing enzyme, wherein the individual primer of the primer set has sequence complementarity with the template nucleic acid molecule, wherein the reaction chamber comprising the reaction mixture is configured to facilitate a nucleic acid amplification reaction with the reaction mixture under conditions sufficient to yield at least one target nucleic acid molecule as an amplification product(s) of the template nucleic acid molecule, wherein the nucleic acid amplification reaction does not yield any amplification product of the control nucleic acid; (b) a sensor array comprising (i) a substrate comprising a plurality of first probes immobilized to a first pixel, a plurality of second probes immobilized to a second pixel, wherein the first probes are configured to capture the individual primer of the primer set, and wherein the second probes are configured to capture the control nucleic acid molecule, and (ii) an array of detectors configured to detect at least one first signal from the first pixel and at least one second signal from the second pixel, wherein a difference between the at least one first signal and the at least one second signal over time is indicative of the individual primer binding with an individual probe of the plurality of first probes; and (c) a computer processor coupled to the sensor array and programmed to (i) subject the reaction mixture to the nucleic acid amplification reaction, and (ii) detect the at least one first signal and the at least one second signal at multiple time points during the nucleic acid amplification reaction. In some embodiments, the computer processor is programmed to detect the template nucleic acid molecule using the difference between the at least one first signal and the at least one second signal. In some embodiments, the reaction mixture comprises a plurality of template nucleic acid molecules, and wherein the first probes specifically bind to a plurality of target nucleic molecules as amplification products of the plurality of the template nucleic acid molecules. In some embodiments, the primer set comprises a plurality of individual primers having different nucleic acid sequences, and wherein the first probes are configured to specifically bind to the plurality of the individual primers. In some embodiments, the array of detectors comprises an optical detector. In some embodiments, the at least one first signal is a first optical signal that is indicative of a first interaction between a first energy acceptor and a first energy donor associated with the individual primer and the individual probe, and wherein the at least one second signal is a second optical signal that is indicative of a second interaction between a second energy acceptor and a second energy donor associated with the control nucleic acid molecule and an additional probe of the second probes. In some embodiments, the optical detector comprises a complementary metal-oxide semiconductor device. In some embodiments, the array of detectors comprises an electrical detector. In some embodiments, the electrical detector comprises a complementary metal-oxide semiconductor device. In some embodiments, the sensor array comprises at least about 100 integrated sensors.

Various techniques and technologies may be used for conducting Q-PCR using a microarray or a CMOS biochip. For example, a number of such techniques are described in U.S. Pat. Nos. 8,048,626, 9,499,861 and 10,174,367, each of which is incorporated herein by reference in its entireties for all purposes

In some embodiments of this invention the light-removable blocking is included in a NA affinity-based detection system such as DNA microarrays. DNA microarrays, which are, essentially, massively parallel affinity-based biosensors, are primarily used to measure gene expression levels, i.e., to quantify the process of transcription of DNA data into messenger RNA molecules (mRNA). The information transcribed into mRNA is further translated to proteins, the molecules that perform most of the functions in cells. Therefore, by measuring gene expression levels, researchers may be able to infer critical information about functionality of the cells or the whole organism. Accordingly, a perturbation from the typical expression levels is often an indication of a disease; thus, DNA microarray experiments may provide valuable insight into the genetic causes of diseases. Indeed, one of the ultimate goals of DNA microarray technology is to allow development of molecular diagnostics and creation of personalized medicine.

A DNA microarray is basically an affinity-based biosensor where the binding is based on hybridization, a process in which complementary DNA strands specifically bind to each other creating structures in a lower energy state. Typically, the surface of a DNA microarray consists of an array (grid) of spots, each containing single stranded DNA oligonucleotide capturing molecules as recognition elements, whose locations are fixed during the process of hybridization and detection. Each single-stranded DNA capturing molecule typically has a length of 25-70 bases, depending on the exact platform and application. In the DNA microarray detection process, the mRNA that needs to be quantified is initially used to generate fluorescent labeled cDNA, which is applied to the microarray. Under appropriate experimental conditions (e.g., temperature and salt concentration), labeled cDNA molecules that are the perfect match to the microarray will hybridize, i.e., bind to the complementary capturing oligos. Nevertheless, there will always be a number of non-specific bindings since cDNA may non-specifically cross-hybridize to oligonucleotide that are not the perfect match but are rather only partial complements (having mismatches). Furthermore, the fluorescent intensities at each spot are measured to obtain an image, having correlation to the hybridization process, and thus the gene expression levels.

Molecular recognition assays generally involve detecting binding events between two types of molecules. The strength of binding can be referred to as “affinity”. Affinities between biological molecules are influenced by non-covalent intermolecular interactions including, for example, hydrogen bonding, hydrophobic interactions, electrostatic interactions and Van der Waals forces. In multiplexed binding experiments, such as those contemplated here, a plurality of analytes and probes are involved. For example, the experiment may involve testing the binding between a plurality of different nucleic acid molecules or between different proteins. In such experiments analytes preferentially will bind to probes for which they have the greater affinity. Thus, determining that a particular probe is involved in a binding event indicates the presence of an analyte in the sample that has sufficient affinity for the probe to meet the threshold level of detection of the detection system being used. One may be able to determine the identity of the binding partner based on the specificity and strength of binding between the probe and analyte.

In developing the solution in the context of DNA microarrays, the invention provides a process whereby (i) cross-hybridization is viewed as interference, rather than noise (akin to wireless communications interference, cross-hybridization actually has signal content); (ii) a model of hybridization and cross-hybridization as a stochastic processes; (iii) use of analytical methods (e.g., melting temperature or Gibbs free energy function) to construct models and use empirical data to fine tune the models; (iv) the detection and quantification of gene expression levels are viewed as a stochastic estimation problem; and (v) construction of optimal estimates. The invention uses statistical signal processing techniques to optimally detect and quantify the targets in microarrays by taking into account and exploiting the above uncertainties.

Various techniques and technologies may be used for synthesizing arrays of biological materials on or in a substrate or support. For example, a number of such techniques are described in U.S. Pat. Nos. 9,223,929 and 9,133,504, each of which is incorporated herein by reference in its entireties for all purposes.

In some embodiments of this invention the light-removable blocking is included in a CMOS biochip system. In some embodiment, the present disclosure provides a fully integrated biosensor array comprising, in order, a molecular recognition layer comprising the NA construct, an optical layer and a sensor layer integrated in a sandwich configuration or in tandem together with additional layers, for example, having another layer inserted between any of the molecular recognition layer, the optical layer and the senor layer. The molecular recognition layer comprises an open surface and a plurality of different probes attached at different independently addressable locations to the open surface. The molecular recognition layer can also transmit light to the optical layer. The optical layer comprises an optical filter layer, wherein the optical layer transmits light from the molecular recognition layer to the sensor layer. The transmittal of light between layers can be filtered by the optical layer. The sensor layer comprises an array of optical sensors that detects the filtered light transmitted through the optical layer. In addition, there can be a fluid volume comprising analyte in fluid contact with the molecular recognition layer. The fluid volume may comprise the NA construct.

An integrated biosensor array of the current disclosure can measure binding of analytes in real-time. An integrated biosensor microarray that can detect binding kinetics of an assay is in contact with an affinity-based assay. The biosensor array comprises a molecular recognition layer comprising binding probes in optical communication a sensor for detecting binding to the probes in real-time.

An integrated fluorescent-based microarray system for real-time measurement of the binding of analyte to a plurality of probes that includes the capturing probe layer, fluorescent emission filter, and image sensor can be built using a standard complementary metal-oxide semiconductor (CMOS) process.

In an embodiment of the invention, the array of optical sensors of the sensor layer is a part of a semiconductor based sensor array. The semiconductor based sensor array can be either an organic semiconductor or an inorganic semiconductor. In some embodiments, the semiconductor device is a silicon-based sensor. Examples of sensors useful in the present invention include, but are not limited to, a charge-coupled device (CCD), a CMOS device, and a digital signal processor. The semiconductor device of the sensor layer can also comprise an integrated in-pixel photocurrent detector. The detector may comprise a capacitive transimpedance amplifier (CTIA).

In another embodiment, the semiconductor device has an in-pixel analog to digital converter. In another embodiment, the array of optical sensors of the sensor layer can be a photodiode array.

The sensor layer can be created using a CMOS process. A semiconductor detection platform can be the assembly of an integrated system capable of measuring the binding events of real-time microarrays (RT-μArrays). In some embodiments, an integrated device system involves a transducer array that is placed in contact with or proximity of the RT-μArray assay.

A semiconductor detection platform for RT-uArrays can include an array of independent transducers to receive and/or analyze the signal from target and probe binding events of a RT-μArray platform. A plurality of transducers can work collectively to measure a number of binding events at any individual microarray spot. For example, transducers dedicated to a spot may add and/or average their individual measured signal.

Detection circuitry connected to an array of optical sensors can be embedded in the sensor layer. Signal processing circuitry can also be connected to the array of optical sensors and embedded in the sensor layer. In some embodiments, the transducers and/or detection circuitry and/or analysis systems are implemented using electronic components which are fabricated and/or embedded in the semiconductor substrate. Examples of such fabrication techniques include, but are not limited to, silicon fabrication processes, micro-electromechanical surface micromachining, CMOS fabrication processes, CCD fabrication processes, silicon-based bipolar fabrication processes, and gallium-arsenide fabrication processes.

The transducer array can be an image sensor array. Examples of such image arrays include, but are not limited to, CMOS image sensor arrays, CMOS linear optical sensors, CCD image sensors, and CCD linear optical sensors. The image sensor can be used to detect the activity of the probe/analyte interaction within the integrated biosensor array platform.

Various techniques and technologies may be used for making and/or using a CMOS biochip system. For example, a number of such techniques are described in U.S. Pat. Nos. 8,637,436 and 8,969,781.

### EXAMPLE 2

### Light-Enabled Nested PCR

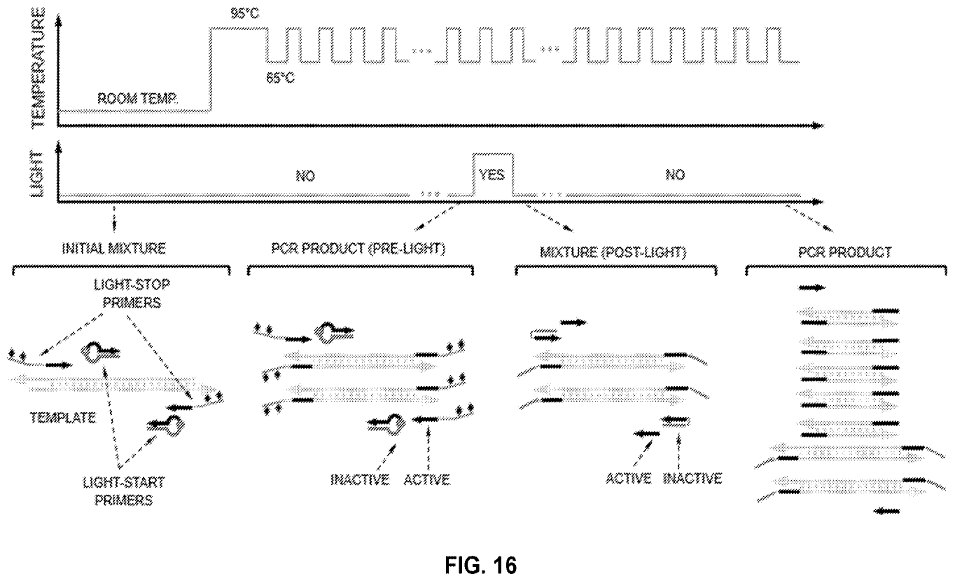

In this example, as depicted in FIG. 16, two pair of primers are used. One pair is light-start while the other is light-stop. At a specific time within the PCR cycles, light is applied to inactivate the light-stop primer pair, and activate the light-start pair.

In some embodiments, the light-stop primer pair flanks the light-start primer pair (see FIG. 16), such that the amplicon generated by the active form of the light-stop primer pair is used as the template for the active form of light-start primer pair. The advantage of this system is that it can increase the specificity and sensitivity of the amplification by reducing non-specific amplicons and products that may be produced due to the amplification of unexpected primer binding sites on the template.

This method, which henceforth can be referred to as “light-enabled nested PCR”, may be an alternative to conventional nested PCR methods where two PCR amplifications are executed in tandem in two different reactions chambers. See G. Bein, R. Gläser, & H. Kirchner, “Rapid HLA-DRB1 genotyping by nested PCR amplification. Tissue antigens,” 1992, 39(2): 68-73; M. Pfeffer, B. Linssen, M. D. Parker, and R. M Kinney, “Specific detection of Chikungunya virus using a RT-PCR/nested PCR combination,” Journal of Veterinary Medicine, Series B, 2002, 49(1): 49-54. The advantage of light-start nested PCR, however, is that both amplification can occur in the same reaction and in a closed tube fashion.

In some embodiments of this invention the light-enabled nested PCR is included in a Q-PCR system. The device, system and method disclosed in Example 1 can be modified and applied herein by using the appropriate NA construct as light-start primer pair and/or light-stop primer pair in the light-enabled nested PCR and radiating the reaction mixture in the process of running the light-enabled nested PCR to start or stop a particular PCR process.

In some embodiments of this invention the light-removable blocking is included in a NA affinity-based detection system such as DNA microarrays.

In some embodiments of this invention the light-removable blocking is included in a CMOS biochip system.

### EXAMPLE 3

### Light-Removable Blocking

In this example, light-stop hybridization probes are used as sequence-selective blockers in polymerase chain reactions or other primer-initiated molecular amplification reactions. See P. L. Dominguez, and M. S. Kolodney, “Wild-type blocking polymerase chain reaction for detection of single nucleotide minority mutations from clinical specimens,” Oncogene, 2005, 24(45): 6830-6834. J. F. Huang, et al., “Single-tubed wild-type blocking quantitative PCR detection assay for the sensitive detection of codon 12 and 13 KRAS mutations,” PloS one, 2015, 10(12).

In some embodiments, the light-stop hybridization probe inhibits the PCR amplification of the wild-type sequence, while allowing the mutant sequence to be synthesized. By doing this the ratio of the wild-type amplicon vs. mutant amplicon decreases, as the amplification progresses. This facilitates better detection of the mutant at the end of the PCR. The presence of the light-stop construct type further allows the removal of the blocker by light to produce clean PCR products with no interfering hybridization probes.

In some embodiments of this invention the light-removable blocking is included in a Q-PCR system. The device, system and method disclosed in Example 1 can be modified and applied herein by using the appropriate NA construct as light-removable blocking probe in tandem with a light-start PCR process, and radiating the reaction mixture in the process of running the light-start PCR to start or stop a particular PCR process.

In some embodiments of this invention the light-removable blocking is included in a NA affinity-based detection system such as DNA microarrays. The device, system and method disclosed in Example 1 can be modified and applied herein by using the appropriate NA construct as light-removable blocking probe in a NA-affinity-based detection system, such as DNA microarrays. When using the NA-affinity-based detection system, for example, to detect a target nucleic acid, the light-removable blocking probe can interact with the target nucleic acid, the immobilized probe, or solution-based probe, or a combination thereof. By radiating the reaction mixture in the process of running the NA affinity-based detection system, different amplicons may be produced and/or different hybridization events may be detected by the NA affinity-based detection system.

In some embodiments of this invention the light-removable blocking is included in a CMOS biochip system. The device, system and method disclosed in Example 1 can be modified and applied herein by using the appropriate NA construct as light-removable blocking probe in a CMOS biochip system. When using the CMOS biochip system, for example, to detect a target nucleic acid, the light-removable blocking probe can interact with the target nucleic acid, the immobilized probe, or solution-based probe, or a combination thereof. By radiating the reaction mixture in the process of running the CMOS biochip system, By radiating the reaction mixture in the process of running the NA affinity-based detection system, different amplicons may be produced and/or different hybridization events may be detected by the CMOS biochip system.

### EXAMPLE 4

### Light-Anchored Primers

In this example, light-stop primers are used to alter the effective length of a primer during PCR.

In some embodiments, the light-stop primer is cleaved into two portions after a specific number of cycles of PCR: An inactive portion derived from the original 5′-terminus of the primer, and an active (extensible) portion derived from the original 3′-end that is capable of continuing PCR after photo-cleavage. This allows for the design of an anchored primer with a high melting temperature (TM) in the initial cycles of PCR. Upon exposure to the light, the length of the primer is shortened both to reduce the TM of the primer and to reduce the length of the resulting amplicon. Applications of this method include the design of a high TM primer to accommodate mismatches within the template in early cycles of PCR and/or to overcome a secondary structure in either an RNA or DNA template.

In some embodiments of this invention the light-anchored primers are included in a Q-PCR system. The device, system and method disclosed in Example 1 can be modified and applied herein by using the appropriate NA construct as light-anchored primers in a light-anchored PCR process. Before exposing to light, the amplicons generated can comprise the full-length of the light-anchored primers. Radiating the reaction mixture can produce a new primer pairs. Each new primer is shorter in length than the corresponding full-length light-anchored primer. Thus, the amplicons produced with the new primer pair can have shorter length than when before exposing to the light. Two sets of amplicons with different lengths can be generated using the same template nucleic acid molecule.

In some embodiments of this invention the light-anchored primers are included in a NA affinity-based detection system such as DNA microarrays.

In some embodiments of this invention the light-anchored primers are included in a CMOS biochip system.

### Other Terms Used in the Present Disclosure

The term “quantitative-PCR” or “Q-PCR,” as used herein generally refers to a polymerase chain reaction (PCR) process that can be used for the qualitative and quantitative determination of nucleic acid sequences. In some cases, Q-PCR is synonymous with real-time PCR. Q-PCR can involve the measurement of the amount of amplification product (or amplicon) as a function of amplification cycle, and use such information to determine the amount of the nucleic acid sequence corresponding to the amplicon that was present in the original sample.

The term “reverse transcription polymerase chain reaction” or “RT-PCR,” as used herein generally refers to a variant of polymerase chain reaction (PCR), in which a ribonucleic acid (RNA) strand is first reverse transcribed into its DNA complement (complementary DNA, or cDNA) using the enzyme reverse transcriptase. The resulting cDNA is subsequently amplified using traditional PCR. RT-PCR utilizes a pair of primers, which are complementary to a defined sequence on each of the two strands of the cDNA. These primers are then extended by a DNA polymerase and a copy of the strand is made after each PCR cycle, leading to exponential amplification. The term “quantitative reverse transcription polymerase chain reaction” or “qRT-PCR,” as used herein, refers to real time detection of a RT-PCR reaction, as similarly done in a Q-PCR reaction.

In the present disclosure, all methods or systems when disclosing for QPCR can be applicable to qRT-PCR after making the corresponding changes as known in the art to a skilled person.

The term “probe” as used herein generally refers to a molecular species or other marker that can bind to a specific target nucleic acid sequence. A probe can be any type of molecule or particle. Probes can comprise molecules and can be bound to the substrate or other solid surface, directly or via a linker molecule.

The term “detector” as used herein generally refers to a device, generally including optical and/or electronic components that can detect signals.

The term “mutation” as used herein generally refers to genetic mutations or sequence variations such as a point mutation, a single nucleotide polymorphism (SNP), an insertion, a deletion, a substitution, a transposition, a translocation, a copy number variation, or another genetic mutation, alteration or sequence variation.

The term “about” or “nearly” as used herein generally refers to within +/- 15%, 10%, 9%, 8%, 7%, 6%, 5%, 4%, 3%, 2%, or 1% of the designated amount.

The term “label” as used herein refers to a specific molecular structure that can be attached to a target molecule, to make the target molecule distinguishable and traceable by providing a unique characteristic not intrinsic to the target molecule.

The term “limiting,” as used herein in the context of a chemical or biological reaction, generally refers to a species that is in a limiting amount (e.g., stoichiometrically limiting) in a given reaction volume such that upon completion of the chemical or biological reaction (e.g., PCR), the species may not be present in the reaction volume.

The term “excess,” as used herein in the context of a chemical or biological reaction, generally refers to a species that is in an excess amount (e.g., stoichiometrically limiting) in a given reaction volume such that upon completion of the chemical or biological reaction (e.g., PCR), the species may be present in the reaction volume.

The term “nucleotide,” as used herein, generally refers a molecule that can serve as the monomer, or subunit, of a nucleic acid, such as deoxyribonucleic acid (DNA) or ribonucleic acid RNA). A nucleotide can be a deoxynucleotide triphosphate (dNTP) or an analog thereof, e.g., a molecule having a plurality of phosphates in a phosphate chain, such as 2, 3, 4, 5, 6, 7, 8, 9, or 10 phosphates. A nucleotide can generally include adenosine (A), cytosine (C), guanine (G), thymine (T) and uracil (U), or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. A nucleotide may be labeled or unlabeled. A labeled nucleotide may yield a detectable signal, such as an optical, electrostatic or electrochemical signal.

A Q-PCR process can be described in the following non-limiting example. A PCR reaction is carried out with a pair of primers designed to amplify a given nucleic acid sequence in a sample. The appropriate enzymes and nucleotides, such as deoxynucleotide triphosphates (dNTPs), are added to the reaction, and the reaction is subjected to a number of amplification cycles. The amount of amplicon generated from each cycle is detected, but in the early cycles, the amount of amplicon can be below the detection threshold. The amplification may be occurring in two phases, an exponential phase, followed by a non-exponential plateau phase. During the exponential phase, the amount of PCR product approximately doubles in each cycle. As the reaction proceeds, however, reaction components are consumed, and ultimately one or more of the components becomes limiting. At this point, the reaction slows and enters the plateau phase. Initially, the amount of amplicon remains at or below background levels, and increases are not detectable, even though amplicon product accumulates exponentially. Eventually, enough amplified product accumulates to yield a detectable signal. The cycle number at which this occurs is called the threshold cycle, or Cₜ. Since the Cₜ value is measured in the exponential phase when reagents are not limited, Q-PCR can be used to reliably and accurately calculate the initial amount of template present in the reaction. The Cₜ of a reaction may be determined mainly by the amount of nucleic acid sequence corresponding to amplicon present at the start of the amplification reaction. If a large amount of template is present at the start of the reaction, relatively few amplification cycles may be required to accumulate enough products to give a signal above background. Thus, the reaction may have a low, or early, Cₜ. In contrast, if a small amount of template is present at the start of the reaction, more amplification cycles may be required for the fluorescent signal to rise above background. Thus, the reaction may have a high, or late, Cₜ. Methods and systems provided herein allow for the measurement of the accumulation of multiple amplicons in a single fluid in a single amplification reaction, and thus the determination of the amount of multiple nucleic acid sequences in the same sample with the methodology of Q-PCR described above.

As used herein in, the term “real-time” generally refers to measuring the status of a reaction while it is occurring, either in the transient phase or in biochemical equilibrium. Real-time measurements are performed contemporaneously with the monitored, measured, or observed ongoing events, as opposed to measurements taken after a reaction is fixed. Thus, a “real time” assay or measurement generally contains not only the measured and quantitated result, such as fluorescence, but expresses this at various time points, that is, in nanoseconds, microseconds, milliseconds, seconds, minutes, hours, etc. “Real-time” may include detection of the kinetic production of signal, comprising taking a plurality of readings in order to characterize the signal over a period of time. For example, a real-time measurement can comprise the determination of the rate of increase or decrease in the amount of an analyte. While the measurement of signal in real-time can be useful for determining rate by measuring a change in the signal, in some cases the measurement of no change in signal can also be useful. For example, the lack of change of a signal over time can be an indication that a reaction (e.g., binding, hybridization) has reached a steady-state.

As used herein, the terms “polynucleotide”, “oligonucleotide”, “nucleotide”, “nucleic acid” and “nucleic acid molecule” generally refer to a polymeric form of nucleotides (polynucleotides) of various lengths (e.g., 20 bases to 5000 kilo-bases), either ribonucleotides (RNA) or deoxyribonucleotides (DNA). This term may refer only to the primary structure of the molecule. Thus, the term may include triple-, double- and single-stranded DNA, as well as triple-, double- and single-stranded RNA. It may also include modifications, such as by methylation and/or by capping, and unmodified forms of the polynucleotide.

Nucleic acids can comprise phosphodiester bonds (i.e. natural nucleic acids). Nucleic acids can comprise nucleic acid analogs that may have alternate backbones, comprising, for example, phosphoramide (see, e.g., Beaucage et al., Tetrahedron 49(10):1925 (1993) and U.S. Pat. No. 5,644,048), phosphorodithioate (see, e.g., Briu et al., J. Am. Chem. Soc. 11 1:2321 (1989), O-methylphosphoroamidite linkages (see, e.g., Eckstein, Oligonucleotides and Analogues: A Practical Approach, Oxford University Press), and peptide nucleic acid (PNA) backbones and linkages (see, e.g., Carlsson et al., Nature 380:207 (1996)). Nucleic acids can comprise other analog nucleic acids including those with positive backbones (see, e.g., Denpcy et al., Proc. Natl. Acad. Sci. USA 92:6097 (1995); non-ionic backbones (see, e.g., U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, (see, e.g., U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook). Nucleic acids can comprise one or more carbocyclic sugars (see, e.g., Jenkins et al., Chem. Soc. Rev. (1995) pp 169-176). These modifications of the ribose-phosphate backbone can facilitate the addition of labels, or increase the stability and half-life of such molecules in physiological environments.

As used herein, the term “amplicon” generally refers to a molecular species that is generated from the amplification of a nucleotide sequence, such as through PCR. An amplicon may be a polynucleotide such as RNA or DNA or mixtures thereof, in which the sequence of nucleotides in the amplicon may correlate with the sequence of the nucleotide sequence from which it was generated (i.e. either corresponding to or complimentary to the sequence). The amplicon can be either single stranded or double stranded. In some cases, the amplicon may be generated by using one or more primers that is incorporated into the amplicon. In some cases, the amplicon may be generated in a polymerase chain reaction or PCR amplification, wherein two primers may be used to produce either a pair of complementary single stranded amplicons or a double-stranded amplicon.

As used herein, the term “probe” generally refers to a molecular species or a marker that can bind to a nucleic acid sequence. A probe can be any type of molecules or particles. Probes can comprise molecules and can be bound to a substrate or a surface, directly or via a linker molecule. As used herein, the singular forms “a”, “an”, and “the” include plural references unless the context clearly dictates otherwise.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-146. (canceled)

147. A nucleic acid construct, comprising:
a) a plurality of nucleotides; and
b) one or more photocleavable moieties,
wherein a photocleavable moiety of said one or more photocleavable moieties is located:
a) at 3′-terminus of said nucleic acid construct;
b) at 5′-terminus of said nucleic acid construct;
c) between said 3′-terminus and said 5′-terminus;
d) on or connected to a nucleobase of a nucleotide of said plurality of nucleotides;
e) on or connected to a ribose of said nucleotide; or
f) between and connected to said nucleotide and another nucleotide of said plurality of nucleotides.

148. The nucleic acid construct of claim 147, wherein said nucleic acid construct is configured to be inactive in a biochemical reaction, wherein said biochemical reaction is a polymerase-catalyzed chain elongation reaction, a polymerase chain reaction (PCR), a reverse transcription polymerase chain reaction (RT-PCR), a ligation, a terminal transferases extension reaction, a hybridization reaction, an exonuclease digest reaction, an endonuclease digest reaction, or a restriction digest reaction.

149. The nucleic acid construct of claim 148, wherein said nucleic acid construct is configured to form a nucleic acid molecule after photocleavage of said one or more photocleavable moieties, and wherein said nucleic acid molecule is configured to be active in said biochemical reaction.

150. The nucleic acid construct of claim 148, wherein said nucleic acid construct is a primer, and wherein said biochemical reaction is said polymerase-catalyzed chain elongation.

151. The nucleic acid construct of claim 150, wherein said one or more photocleavable moieties are located at said 3′-terminus.

152. The nucleic acid construct of claim 150, wherein said photocleavable moiety is located between said 3′-terminus and said 5′-terminaus and on a nucleobase.

153. The nucleic acid construct of claim 150, wherein said photocleavable moiety is located between said 3′-terminus and said 5′-terminaus and between said two consecutive members of said plurality of nucleotides.

154. The nucleic acid construct of claim 153, wherein said 3′-terminus is configured to be inactive in said biochemical reaction.

155. The nucleic acid construct of claim 147, wherein said nucleic acid construct comprises a first nucleic acid section and a second nucleic acid section complementary to said first nucleic acid section, wherein said nucleic acid construct is configured to form a hairpin structure.

156. The nucleic acid construct of claim 155, wherein said first nucleic acid section and said second nucleic acid section do not comprise said one or more photocleavable moieties.

157. A method of conducting a polymerase-catalyzed chain elongation reaction, comprising:
a) providing a reaction mixture comprising a nucleic acid construct and a template nucleic acid molecule, wherein said nucleic acid construct has sequence complementary with said template nucleic acid molecule, and wherein said nucleic acid construct comprises:
i) a plurality of nucleotides having a sequence that is complementary to said template nucleic acid molecule; and
ii) one or more photocleavable moieties;
wherein a photocleavable moiety of said one or more photocleavable moieties is located:
i) at 3′-terminus of said nucleic acid construct;
ii) at 5′-terminus of said nucleic acid construct;
iii) between said 3′-terminus and said 5′-terminus;
iv) on or connected to a nucleobase of a nucleotide of said plurality of nucleotides;
v) on or connected to a ribose of said nucleotide; or
vi) between and connected to said nucleotide and another nucleotide of said plurality of nucleotides; and
b) radiating said reaction mixture or said nucleic acid construct with light to yield said nucleic acid construct hybridized to said template nucleic acid molecule, thereby initiating said polymerase-catalyzed chain elongation.

158. The method of claim 157, further comprising, subsequent to (b), using said nucleic acid construct hybridized to said template nucleic acid molecule to generate a growing strand complementary to said template nucleic acid molecule.

159. The method of claim 158, further comprising detecting an optical signal from a nucleotide incorporated into said growing strand.

160. The method of claim 158, further comprising detecting a decrease in intensity of an optical upon formation of said growing strand.

161. The method of claim 158, further comprising detecting an increase in intensity of an optical upon formation of said growing strand.

162. The method of claim 157, wherein said reaction mixture is in contact with a surface of an array comprising a plurality of nucleic acid probes at a plurality of independently addressable locations on said surface.

163. The method of claim 162, further comprising measuring hybridization of two or more amplicons of said polymerase-catalyzed chain elongation reaction with said plurality of nucleic acid probes.

164. The method of claim 157, wherein said nucleic acid construct remains intact in said reaction mixture prior to said radiating in b).

165. The method of claim 157, further comprising, subsequent to said radiating in b), performing said polymerase-catalyzed chain elongation reaction using said nucleic acid construct hybridized to said template nucleic acid molecule as a primer.

166. The method of claim 164, wherein said reaction mixture further comprises another primer, wherein said another primer is active in said polymerase-catalyzed chain elongation reaction.

167. The method of claim 165, wherein said another primer is active in said polymerase-catalyzed chain elongation prior to said radiating in b).

168. The method of claim 165, wherein said polymerase-catalyzed chain elongation reaction in b) produces an amplicon comprising said another primer.

169. The method of claim 168, wherein said amplicon comprises a quencher.

170. The method of claim 157, wherein said reaction mixture further comprises a liming primer and an excess primer, wherein said nucleic acid construct comprises said limiting primer or said excess primer.


SEQUENCE LISTING (13 sequences)

SEQ ID NO: 1 - DNA, 20 bp, Artificial Sequence
  Description of Artificial Sequence Synthetic oligonucleotide
ctcggtcgtccaatatcgaa

SEQ ID NO: 2 - DNA, 20 bp, Artificial Sequence
  Description of Artificial Sequence Synthetic oligonucleotide
ctcggtcgtccaatatcgaa

SEQ ID NO: 3 - DNA, 20 bp, Artificial Sequence
  Description of Artificial Sequence Synthetic oligonucleotide
ctcggtcgtccaatatcgaa

SEQ ID NO: 4 - DNA, 20 bp, Artificial Sequence
  Description of Artificial Sequence Synthetic oligonucleotide
ctcggtcgtccaatatcgaa

SEQ ID NO: 5 - DNA, 12 bp, Artificial Sequence
  Description of Artificial Sequence Synthetic oligonucleotide
ctcggtcgtcca

SEQ ID NO: 6 - DNA, 20 bp, Artificial Sequence
  Description of Artificial Sequence Synthetic oligonucleotide
ctcggtcgtccaatatcgaa

SEQ ID NO: 7 - DNA, 14 bp, Artificial Sequence
  Description of Artificial Sequence Synthetic oligonucleotide
cgtccaatatcgaa

SEQ ID NO: 8 - DNA, 13 bp, Artificial Sequence
  Description of Artificial Sequence Synthetic oligonucleotide
gtccaatatcgaa

SEQ ID NO: 9 - DNA, 13 bp, Artificial Sequence
  Description of Artificial Sequence Synthetic oligonucleotide
accgttaggatgc

SEQ ID NO: 10 - DNA, 16 bp, Artificial Sequence
  Description of Artificial Sequence Synthetic oligonucleotide
gcatcctaacggttaa

SEQ ID NO: 11 - DNA, 7 bp, Artificial Sequence
  Description of Artificial Sequence Synthetic oligonucleotide
accgtta

SEQ ID NO: 12 - DNA, 13 bp, Artificial Sequence
  Description of Artificial Sequence Synthetic oligonucleotide
gcatcctaacggt

SEQ ID NO: 13 - DNA, 16 bp, Artificial Sequence
  Description of Artificial Sequence Synthetic oligonucleotide
aataccgttaggatgc